SEQUENCE LISTING (15 sequences)

SEQ ID NO: 1 - DNA, 21 bp, Mus musculus
agtccctgccctttgtacaca

SEQ ID NO: 2 - DNA, 19 bp, Mus musculus
cgatcccagggcctcacta

SEQ ID NO: 3 - DNA, 21 bp, Mus musculus
atggcgaacttcacacctgtc

SEQ ID NO: 4 - DNA, 21 bp, Mus musculus
ctgtcgcaatgaacaccatct

SEQ ID NO: 5 - DNA, 18 bp, Mus musculus
tgcacccaaaccgaagtc

SEQ ID NO: 6 - DNA, 20 bp, Mus musculus
gtcagaagccagcgttcacc

SEQ ID NO: 7 - DNA, 20 bp, Mus musculus
attgggatcatcttgctggt

SEQ ID NO: 8 - DNA, 19 bp, Mus musculus
cctgctgttcacagttgcc

SEQ ID NO: 9 - DNA, 19 bp, Mus musculus
tgatccctgcaggtccaca

SEQ ID NO: 10 - DNA, 20 bp, Mus musculus
ctgcgagtgcattccgctta

SEQ ID NO: 11 - DNA, 20 bp, Mus musculus
gtgaagggtacaagggggtg

SEQ ID NO: 12 - DNA, 20 bp, Mus musculus
acatctcagcaacccacaca

SEQ ID NO: 13 - skipped

SEQ ID NO: 14 - DNA, 20 bp, Artificial Sequence
  A synthetic sequence
gtgstgcayggytgtcgtca

SEQ ID NO: 15 - DNA, 20 bp, Artificial Sequence
  A synthetic sequence
acgtcrtccmcaccttcctc

(19) United States
(12) Patent Application Publication
(10) Pub. No.: US 2025/0302831 A1
(43) Pub. Date: Oct. 2, 2025

(54) THERAPY FOR ALCOHOL-RELATED LIVER DISEASE

(71) Applicant: THE REGENTS OF THE UNIVERSITY OF CALIFORNIA, Oakland, CA (US)

(72) Inventors: Bernd Schnabl, San Diego, CA (US); Ana Cristina Llorente lzquierdo, San Diego, CA (US)

(21) Appl. No.: 18/287,646

(22) PCT Filed: Apr. 20, 2022
(86) PCT No.: PCT/US22/25558

§ 371 Date: Oct. 19, 2023

Related U.S. Application Data
(60) Provisional application No. 63/177,316, filed on Apr. 20, 2021.

(51) Int. Cl. A61K 31/5025; A61K 31/27; A61K 31/404; A61K 31/426; A61K 31/4365; A61K 31/437; A61K 31/439; A61K 31/4436; A61K 31/4439; A61K 31/444; A61K 31/4545; A61K 31/497; A61K 31/501; A61K 31/517; A61K 31/519; A61K 31/55; A61K 31/551; A61P 1/16

(52) U.S. Cl. CPC A61K 31/5025; A61K 31/27; A61K 31/404; A61K 31/426; A61K 31/4365; A61K 31/437; A61K 31/439; A61K 31/4436; A61K 31/4439; A61K 31/444; A61K 31/4545; A61K 31/497; A61K 31/501; A61K 31/517; A61K 31/519; A61K 31/55; A61K 31/551; A61P 1/16

(57) ABSTRACT

A method to prevent, inhibit or treat liver disease in a mammal, comprising administering to the mammal an effective amount of a composition comprising one or more AChR4 positive allosteric modulators, is provided.

### CROSS-REFERENCE TO RELATED APPLICATIONS

This application is a U.S. National Stage Filing under 35 U.S.C. 371 from International Application No. PCT/US2022/025558, filed on Apr. 20, 2022, and published as WO 2022/226078 A1 on Oct. 27, 2022 which claims the benefit of the filing date of U.S. application No. 63/177,316, filed on Apr. 20, 2021, the disclosure of each of which is incorporated by reference herein.

### INCORPORATION BY REFERENCE OF SEQUENCE LISTING

This application contains a Sequence Listing which has been submitted electronically in ST25 format and hereby incorporated by reference in its entirety. Said ST25 file, created on May 6, 2024, is name 1133095US1.txt and is 2,456 bytes in size.

### BACKGROUND

Alcohol consumption is the seventh leading risk factor for death worldwide (Collaborators, 2018) and alcoholic liver disease (ALD) is the major cause of liver transplantation in the West (Lee et al., 2019). Alcohol metabolites can directly damage the liver, but the gut-liver axis may also control ALD pathogenesis through complex and obscure mechanisms. IL-6 is one of several cytokines elevated in ALD that affects the liver and the intestine (Hong et al., 2002). Although IL-6 expression correlates with disease severity (Sheron et al., 1991), IL-6 also exerts barrier protective effects (Kuhn et al., 2018).

### SUMMARY

Although alcohol intake is associated with increased intestinal permeability, it is not clear how the intestinal immune system affects liver disease. Paradoxically, chronic alcohol use increases intestinal goblet cell (GC) number and mucin production in patients and mice. However, this comes with the expense of GC associated antigen passages (GAPs) closure and defective delivery of luminal antigens and bacteria to lamina propria (LP) antigen presenting cells (APCs).

As disclosed herein, GAPs are controlled by an intestinal IL-6 signal transducer (IL6ST/gp130). Specifically, administering a muscarinic AChR positive allosteric regulator (e.g., a mAChR4 PAM), VU0467154, stimulated intestinal GAP formation in mice. WT mice fed an ethanol containing diet for 10 weeks, were treated with VU0467154 during the last 29 days. VU0467154 has no gastrointestinal motility side effects and excellent oral bioavailability. VU0467154 treatment reduced ethanol-induced GAP closure, and protected mice from ethanol-induced liver injury, steatosis and inflammation. mAChR4 PAM treatment did not affect intestinal ethanol absorption, as pairfed mice had similar blood alcohol levels. Frequencies of LP-APCs (CD11c+, MHCII+) and CD103+ CD11b+ DC populations in ethanol fed mice were increased by VU0467154 treatment, which also induced intestinal Reg3g, Reg3b and IL-10 expression and prevented ethanol-mediated bacterial translocation to MLN and Liver. The results indicate that pharmacological manipulation of mAChR4 reduced ethanol-induced steatohepatitis. Thus, mAChR4 positive allosteric modulators (PAMs) stimulate intestinal GAP formation, thereby increasing tolerogenic LP-APCs and Reg3 expression, which prevents microbial translocation and protects against alcoholic liver disease (ALD), e.g., alcoholic steatohepatitis. Since IL6ST signaling modulates intestinal immunity through mAChR4, GAP induction by mAChR4 PAMs is a strategy for enhancing intestinal immune tolerance and interception with ALD and other diseases linked to uncontrolled microbial translocation.

In one embodiment, the disclosure provides methods of preventing, inhibiting or treating liver disease and other diseases linked to uncontrolled microbial translocation. In one embodiment, the method includes administering to a mammal in need thereof a composition having one or more mAChR4 PAMs, in an amount effective to prevent, inhibit or treat liver disorders or other diseases linked to uncontrolled microbial translocation. In one embodiment, a single dose may show activity. In one embodiment, the composition is systemically administered, e.g., orally administered.

In one embodiment, the composition for use in the methods has one or more compounds that are AChR PAMs, including but not limited to VU0152100, LY2033298, VU6013720, VU6021302, VU6021625, LY2119620, VU0467485, VU10010, compounds disclosed in WO2017021728, the disclosure of which is incorporated by reference herein, McN-A-343 (C7041), Xanomeline (X2754), Thiochrome, Vanderbilt's VU0010010, LY2033298, LY2119620, VU0152099, ML173, VU0448088 [ML253], VU0467154, VU0467485/AZ13713945, VU0409524, VU6002703, VU6003130, VU6005877, ([11C]MK-6884], MK-4710, CVL-231 NCT04136873, VU0238441, HTL-9936, dihydroquinazolinone derivates such as those disclosed in “Discovery of dihydroquinazolinone derivatives as potent, selective, and CNS-penetrant M1 and M4 muscarinic acetylcholine receptors agonists.” (Bioorg. Med. Chem. Lett. 2015, 25, 5357-536), the disclosure of which is incorporated by reference herein, N-substituted 7-azaindoline derivates such as those disclosed in Suwa et al. (Discovery of N-sulfonyl-7-azaindoline derivatives as potent, orally available and selective M4 muscarinic acetylcholine receptor agonists. Bioorg. Med. Chem. Lett. 2014, 24, 2909-2912), the disclosure of which is incorporated by reference herein, N-substituted oxindoles such as those disclosed in Sumiyoshi et al. (Discovery of novel N-substituted oxindoles as selective M1 and M4 muscarinic acetylcholine re-ceptors partial agonists. ACS Med. Chem. Lett. 2013, 4, 244-248), the disclosure of which is incorpaoited by reference herein, HTL0016878, pyrazine, 1,2,3-thiadiazoles, or clozapoine. In one embodiment, the compound comprises PT-1148 hM4 (PAM EC50=3 nM), [11C]MK-6884, MK-4710 (IC50=17 nM), PT-6950 hM4 (EC50=20 nM), LY2033298 (hM4 with an EC50 of 8 to 41 nM), PT-3763 hM4 (PAM EC50=45 nM), VU0448088 [ML253](hM4 EC50=56 nM, VU0467485/AZ13713945 hM4 (EC50=78.8 nM), VU0152100 human (EC50=95 nM) or CVL-231.

In one embodiment, a method to prevent, inhibit or treat liver disease in a mammal, comprising administering to the mammal an effective amount of a composition comprising one or more AChR4 positive allosteric modulators, is provided. In one embodiment, the mammal is a human. In one embodiment, the composition is systemically administered. In one embodiment, the composition is orally administered. In one embodiment, the composition is parenterally administered. In one embodiment, the modulator comprises VU0467154, VU0152100, VU0152099, LY2033298, or VU010010. In one embodiment, the composition is a sustained realse composition. In one embodiment, the mammal has alcoholic liver disease. In one embodiment, one or multiple doses of a Gp130 agonist are administered. In one embodiment, the Gp130 agonist comprises an antibody. In one embodiment, the Gp130 agonist comprises CAS339303-87-6 (UCLA GP130 2). In one embodiment the Gp130 agonsit comprises a compound disclosed in https://www.nature.com/articles/s4l586-019-1601-9, which is incorporated by reference herein, e.g., IC7, a chimeric cytokine that protects against metabolic disease (from Findeisen et al., Nature, 2019)], or a gp130-cytokine such as CNTF, LIF, OSM, CLC, CT-1, IL-6 and IL-11, CLC/NN-1, sIL-6R, CLCF1, CLCF1 variants (L86F, Q96R, and H148R), UCLA GP130 2 or GP130 receptor agonist-1. In one embodiment, the amount reduces ethanol-induced steatohepatitis. In one embodiment, the amount reduces ethanol-induced liver injury. In one embodiment, the amount reduces steatosis.

In one embodiment, a method to prevent, inhibit or treat microbial translocation in a mammal, comprising administering to the mammal an effective amount of a composition comprising one or more AChR4 positive allosteric modulators, is provided. In one embodiment, the mammal is a human. In one embodiment, the composition is systemically administered. In one embodiment, the composition is orally administered. In one embodiment, the composition is parenterally administered. In one embodiment, the modulator comprises VU0467154, VU0152100, VU0152099, LY2033298, or VU010010. In one embodiment, the composition is a sustained release composition. In one embodiment, the mammal has alcoholic liver disease. In one embodiment, multiple doses of a Gp130 agonist are also administered. In one embodiment, the Gp130 agonist comprises an antibody. In one embodiment, the Gp130 agonist comprises CAS339303-87-6 (UCLA GP130 2). In one embodiment, the amount reduces ethanol-induced steatohepatitis. In one embodiment, the amount reduces ethanol-induced liver injury. In one embodiment, the amount reduces steatosis.

In one embodiment, a method to stimulate intestinal GAP formation or increase tolerogenic LP-APCs and Reg3 expression in a mammal is provided, comprising administering to the mammal an effective amount of a composition comprising one or more AChR4 positive allosteric modulators. In one embodiment, the mammal is a human. In one embodiment, the composition is systemically administered. In one embodiment, the composition is orally administered. In one embodiment, the composition is parenterally administered. In one embodiment, the modulator comprises VU0467154, VU0152100, VU0152099, LY2033298, or VU010010. In one embodiment, the composition is a sustained release composition. In one embodiment, the mammal has alcoholic liver disease. In one embodiment, multiple doses of the administering a Gp130 agonist. In one embodiment, the Gp130 agonist comprises an antibody. In one embodiment, the Gp130 agonist comprises CAS 339303-87-6 (UCLA GP130 2). In one embodiment, the amount reduces ethanol-induced steatohepatitis. the amount reduces ethanol-induced liver injury. In one embodiment, the amount reduces steatosis.

In one embodiment, a method to enhance intestinal immune tolerance in a mammal, comprising administering to the mammal an effective amount of a composition comprising one or more AChR4 positive allosteric modulators is provided. In one embodiment, the mammal is a human. In one embodiment, the composition is systemically administered. In one embodiment, the composition is orally administered. In one embodiment, the composition is parenterally administered. In one embodiment, the modulator comprises VU0467154, VU0152100, VU0152099, LY2033298, or VU010010. In one embodiment, the composition is a sustained release composition. In one embodiment, the mammal has alcoholic liver disease. In one embodiment, multiple doses of a Gp130 agonist are also administered. In one embodiment, the Gp130 agonist comprises an antibody. In one embodiment, the Gp130 agonist comprises CAS 339303-87-6 (UCLA GP130 2). In one embodiment, the amount reduces ethanol-induced steatohepatitis. In one embodiment, the amount reduces ethanol-induced liver injury. In one embodiment, the amount reduces steatosis.

### BRIEF DESCRIPTION OF THE FIGURES

FIGS. 1A-1I. Chronic ethanol alters proximal intestinal GC in mice and humans. (A-B and E-H) C57BL/6 WT mice were fed control (n=6-9) or ethanol containing Lieber DeCarli diets (n=6-8) for 10 weeks. (A) Percentage of Muc2 positive stained area. (B) Representative sections of Muc2 (red) showing GCs and DAPI (blue) immunofluorescence staining. Scale bar=250 μm. (C, D) Periodic Acid/Schiff (PAS) staining of paraffin-embedded small intestinal sections of duodenal biopsies from non-alcoholic controls (n=8) and patients with AUD (n=15). (C) Positive cells were enumerated in each villus and crypt. (D) Representative PAS stained sections showing increased number of GCs in the duodenum of AUD patients as compared with controls. Scale bar=100 μm. (E) To study GC GAP formation, a 2 cm loop in the small intestine was injected with tetramethylrhodamine (TMR) dextran and GAPs were counted as dextran-filled columns traversing the nucleated epithelium and positive for Muc2 staining. Number of GAPs per villus was quantified. (F) Representative sections stained with TMR-dextran (red) showing GAPs, Muc2 antibody (green) showing GCs and DAPI (blue) marking nuclei. Scale bar=25 μm. (G) Chrm4 mRNA was quantified by qPCR. (H) E. faecalis genetically modified with an EGFP vector (5×10⁹ CFUs) were gavaged 9 hours and 1 hour before euthanasia (n=6). Representative sections stained with Muc2 antibody (red), EGFP (green) and DAPI (blue). Scale bar=50 μm. (i) EGFP-E. faecalis (5×10⁹ CFUs) were gavaged at 3 and 0.5 hours before euthanasia (n=6). Representative section of EGFP (green), TMR-dextran (red) and DAPI (blue) stained intestinal sections. Left panel: scale bar=100 μm, and right panel (amplification of the white discontinuous oval): scale bar=12 μm. (F and I) Arrowheads indicate GAPs. P value was determined by two-sided unpaired Student t test or Mann-Whitney U-statistic test. Results are expressed as mean±s.e.m. *P<0.05.

FIGS. 2A-2Q. Expression of activated gp130 in IECs prevents ethanol-induced liver disease and promotes GAP formation. (A) Chrm4 mRNA in small intestinal organoids from WT mice (n=2) was quantitated by qPCR after stimulation with IL-6 (20 ng/ml) in the presence of ethanol (10 mM) for 12 hours. 6 technical replicates were pooled in each of two independent biological experiments. (B) mAChR4 protein amounts in WT small intestinal organoids after stimulation with (n=4) or without IL-6 (20 ng/ml) (n=6) in the presence of ethanol for 12 hours. 6 technical replicates were pooled in each independent biological experiment. (C) Representative mAChR4 immunoblots. (d-q) WT mice and gp130^{Act/IEC} littermates were fed control (n=4-15) or ethanol containing Lieber DeCarli diets (n=4-44) for 10 weeks. (D) Plasma levels of ALT. (E) Hepatic triglyceride content. (F) Representative H&E stained liver sections. Scale bar=200 μm. (G-H) Hepatic Cxcl1 and Ccl2 mRNAs were quantitated by qPCR. (I) Percentage of Muc2 positive stained area. (J) Representative sections stained with Muc2 antibody (red) showing GCs and DAPI (blue). Scale bar=200 μm. (K) Number of GAPs per villus. (L) Representative TMR-dextran (red) showing GAPs, Muc2 antibody (green) and DAPI (blue) stained small intestine sections showing GAPs (Arrowheads). Scale bar=25 μm. (M) Small intestinal Chrm4 mRNA was quantified by qPCR. (N) mAChR4 protein amounts relative to β-actin in isolated GCs. (O) Immunoblots of mAChR4 and β-actin in isolated goblet cells. (P) Total fecal bacteria were quantitated by qPCR. (Q) Principal coordinate analysis (PCoA) with weighted UniFrac of 16S rRNA sequencing of fecal samples. P values were determined by One-way ANOVA with Tukey's post-hoc test (D, E, I, K and pP), by two-sided unpaired Student t test or Mann-Whitney U-statistic test (A, B, G, H, M and N) and by two-sided paired t test (M). Results are expressed as mean±s.e.m. *P<0.05.

FIGS. 3A-3T. Expression of activated gp130 in IECs stimulates protective intestinal adaptive immune response via mAChR4-mediated GAP formation. (A-T) WT mice and gp130^{Act/IEC} littermates were fed control (n=4-11) or ethanol containing Lieber DeCarli diets (n=6-17) for 10 weeks. (A-D, J-T) A group of littermate mice were treated with the mAChR antagonist tropicamide (20 mg/kg) during the last 29 days (n=5-17). (A-C) Isolated LP immune cells for APC subset identification studies were stimulated with flagellin (100 ng/ml) for 2.5 hours. (A-B) Frequencies of tolerogenic APC subsets in total mononuclear phagocyte population were gated according to CD45, MHCII, CD11c, CD103, CD11b, and CX3CR1 expression. (c) Total number of IL-23⁺ cells in all APCs subsets. (D) Frequencies of ILC3 (CD45⁺, CD3⁻, ROR_{y}t⁺) cells after stimulation of isolated LP leukocytes with mouse IL-23 (40 ng/μl) for 4 hours. (E) Representative ILC3 and (F) IL-22 expressing ILC3 plots. (G-H) Quantification of Reg3g and Reg3b protein amounts relative to total protein identified by stain-free imaging technology. (I) Representative Reg3g and Reg3b immunoblots. (J, K) Number of colony forming units (CFU) of anaerobically cultured bacteria from sterile collected mesenteric lymph nodes and liver. (L) Number of GAPs per villus. (M) Representative TMR-dextran (red), Muc2 antibody (green) and DAPI (blue) stained small intestinal sections showing GAPs (Arrowheads). Scale bar=25 μm. (N) Plasma ALT concentrations. (O) Quantification of the Oil red O (ORO)-stained liver sections. (P) Representative H&E stained liver sections. Scale bar=200 μm. (q) Hepatic Cxcl1 mRNA quantitated by qPCR. (R, S) Reg3g and Reg3b proteins relative to total protein identified by stain-free imaging technology. (T) Representative Reg3g and Reg3b immunoblots. P values were determined by two way (A-D, J-K and O) or one-way ANOVA (F, L, N and Q) with Tukey's post-hoc test and by two sided unpaired Student t test or Mann-Whitney U-statistic test (g, H, R and S). Results are expressed as mean±s.e.m. *P<0.05.

FIGS. 4A-4Q. A mAChR4 PAM reduces ethanol-induced liver disease. WT mice were fed control (n=3-9) or ethanol containing Lieber DeCarli diets (n=4-38) for 10 weeks in the presence or absence of the specific mAChR4 PAM VU0467154 (5 mg/Kg), dissolved in the diet during the last 29 days. (A) Number of GAPs per villus. (B) Representative TMR-dextran (red), Muc2 (green) and DAPI (blue) stained small intestine sections showing GAPs (Arrowheads). Scale bar=25 μm. (C) Plasma ALT concentrations. (D) Representative H&E stained liver sections. Scale bar=200 μm. (E) Quantification of ORO-stained liver sections. (F) Representative oil ORO-stained liver sections. Scale bar=100 μm. (G) Hepatic Cxcl1 mRNA amounts. (H-K) DCs were stimulated with flagellin (100 ng/ml) for 2.5 hours before staining. (H) Frequencies of CD45⁺, MHCII⁺, CD11c⁺ APCs in total mononuclear phagocyte population. (I) Representative APCs plots. (J) Frequencies of CD45⁺, MHCII⁺, CD11c⁺, CD103⁺, CD11b⁺ DCs. (K) Frequencies of tolerogenic APC subsets according to CD45, MHCII, CD11c, CD103, CD11b and CX3CR1 expression. (L-N) Quantification of Reg3g, Reg3b and IL-10 proteins relative to total protein identified by stain-free imaging technology. (O) Representative Reg3g, Reg3b, and IL-10 immunoblots. (P, Q) Number of CFUs of anaerobically cultured bacteria from sterile collected MLN and liver. P values were determined by one-way ANOVA (A, C, E, G, H, J, P, Qq) with Tukey's post-hoc test and by two-sided unpaired Student t test or Mann-Whitney U-statistic test (K, L-N). Results are expressed as mean±s.e.m. *P<0.05.

FIGS. 5A-5E. Food intake and plasma ethanol level in mice expressing active gp130 in IECs after treatments. (A-C) WT mice and their gp130^{Act/IEC} littermates were fed an oral control diet (n=5-7) or ethanol containing Lieber DeCarli diet (n=11-27) for 10 weeks. (A-B) A group of littermate mice were treated with the mAChR4 inhibitor tropicamide (20 mg/kg) during the last 29 days as interventional approach (n=8-19). (A) Food intake of liquid diet. (B) Levels of 679 ethanol in plasma. (C, D) Representative images of isolated goblets cell fraction. (C) Scale bar=680 100 μm, (D) Scale bar=20 μm. (E) Representative sections of mAChR4 (green) and DAPI (blue) immunofluorescence staining showing distribution of the receptors in goblet cells. Scale bar=20 μm. P values were determined by two-way (A) or one-way ANOVA (B) with Tukey's post-hoc test. Results are expressed as mean±s.e.m. *P<0.05.

FIGS. 6A-6E. Expression of active gp130 in IECs regulates LP-APCs in small intestine. (A-D) WT mice and their gp130^{Act/IEC} littermates were fed an oral control diet (n=10-11) or ethanol containing Lieber DeCarli diet (n=11-12) for 10 weeks. A group of littermate mice were treated with mAChR4 inhibitor tropicamide (20 mg/kg) during the last 29 days as interventional approach (n=5-11). LP immune cells were isolated and stimulated with flagellin (100 ng/ml) for 2.5 hours. (A) Frequencies of APCs (CD45⁺, MHCII⁺, CD11c⁺) at the gate. (B) Representative APCs plots. (C) Total number of APCs (CD45⁺, MHCII⁺, CD11c⁺). (D) Total numbers of subsequently gated APCs (CD45⁺, MHCII⁺, CD11c⁺) according to CD103, CD11b and CX3CR1 expression markers. (E) Total number of IL-10 expressing cells from all the APCs subsets. P values were determined by two-way ANOVA with Tukey's post-hoc test. Results are expressed as mean±s.e.m. *P<0.05.

FIGS. 7A-7E. Expression of active gp130 in IECs regulates LP-Tregs in small intestine. (A-D) WT mice and their gp130^{Act/IEC} littermates were fed an oral control diet (n=5-8) or ethanol containing Lieber DeCarli diet (n=8-12) for 10 weeks. A group of littermate mice were treated with mAChR4 inhibitor tropicamide (20 mg/kg) during the last 29 days as interventional approach (n=5-11). (A-C) Cells were stimulated with PMA (10 ng/ml) plus ionomycin (500 ng/ml) for 4 hours. (A) Frequencies at the gate of Treg cells (CD4⁺, CD25⁺ and FOXP3⁺). (b) Representative Treg plots. (C) Total numbers of Treg cells (CD4⁺, CD25⁺ and FOXP3⁺). (D) Quantification of IL-10 protein levels relative to total protein identified with stain-free imaging technology. (E) Representative IL-10 blots. P values were determined by two-way ANOVA with Tukey's post-hoc test (A and C) and by two-sided unpaired Student t test or Mann-Whitney U-statistic test (D). Results are expressed as mean±s.e.m. *P<0.05.

FIGS. 8A-8E. Tropicamide-mediated inhibition of small intestinal GAPs reverts the protection against ethanol-induced liver injury in gp130^{Act/IEC} mice. (A-E) WT mice and their gp130^{Act/IEC} littermates were fed an oral control diet (n=5-15) or ethanol containing Lieber DeCarli diet (n=4-44) for 10 weeks. (A-I) A group of littermate mice were treated with the mAChR4 inhibitor tropicamide (20 mg/kg) during the last 29 days as interventional approach (n=6-17). (A-B) Representation of already described data, now showing all groups in one graph for comparison. (A) Number of GAPs per villus. (B) Plasma levels of ALT. (C) Representative ORO-stained liver sections. Scale bar=100 μm. (D-E) Hepatic Ccl2 and Cxcl5 mRNA expression by qPCR. P values were determined by two-way (A and B) or one-way ANOVA (D-E) with Tukey's post-hoc test. Results are expressed as mean±s.e.m. *P<0.05.

FIGS. 9A-9B. Food intake and plasma ethanol level in mice after mAChR4 PAM treatment. (A-B) WT mice were fed an oral control diet (n=7-11) or ethanol containing Lieber DeCarli diet (n=7-27) for 10 weeks in the presence or absence of a specific mAChR4 positive allosteric modulators, VU0467154 (5 mg/Kg), dissolved in the diet during the last 29 days as interventional approach. (A) Food intake of liquid diet. (B) Levels of ethanol in plasma. P values were determined by one-way ANOVA (A) with Tukey's post-hoc test and by two-sided unpaired Student t test or Mann-Whitney U-statistic test (B). Results are expressed as mean±s.e.m. *P<0.05.

FIG. 10. Graphical abstract. Upon mucin secretion, goblet cells (GCs) form goblet cell associated antigen passages (GAPs) in response to acetylcholine (ACh) acting on muscarinic ACh receptor 4 (mAChR4). GAPs deliver luminal antigens and bacteria to subjacent tolerogenic CD103⁺, CD11b⁺, CX3CR1⁻ DC subset and CD103⁻, CD11b⁺, CX3CR⁺ APCs. Training of these specific LP-APC subsets by GAPs-mediated delivered antigens induces IL-23 secretion which in turn triggers IL-22 production by type 3 innate lymphoid cells (ILC3) and stimulation of Reg3 antimicrobial peptides. This particular adaptive immune response promotes an antibacterial defense, prevents ethanol-induced bacterial translocation to the liver and ameliorates ethanol-induced liver disease. Ethanol-induced suppression of mAChR4 and the mAChR4 antagonist tropicamide inhibit GAP formation. IL6ST signaling and the mAChR4 positive allosteric modulator (PAM), VU0467154, stimulate GAP formation, which offers a promising option to prevent the progression of alcohol-related liver disease through regulation of the intestinal immune response.

### DETAILED DESCRIPTION

### Definitions

A composition is comprised of “substantially all” of a particular compound, or a particular form of a compound (e.g., an isomer) when a composition comprises at least about 90%, and at least about 95%, 99%, and 99.9%, of the particular composition on a weight basis. A composition comprises a “mixture” of compounds, or forms of the same compound, when each compound (e.g., isomer) represents at least about 10% of the composition on a weight basis. A AChR4 PAM, e.g., VU0467154, can be prepared as an acid salt or as a base salt, as well as in free acid or free base forms. In solution, certain of the compounds may exist as zwitterions, wherein counter ions are provided by the solvent molecules themselves, or from other ions dissolved or suspended in the solvent.

It will be understood that when compounds contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

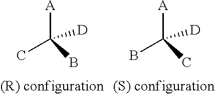

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. In one embodiment, the isolated isomer is at least about 80%, e.g., at least 90%, 98% or 99% pure, by weight.

Isolated optical isomers may be purified from racemic mixtures by well-known chiral separation techniques. According to one such method, a racemic mixture of a compound of the disclosure, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present methods can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, PA, p. 1418 (1985), the disclosure of which is hereby incorporated by reference.

The compounds described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The following definitions are used, unless otherwise described: halo or halogen is fluoro, chloro, bromo, or iodo. Alkyl, alkoxy, alkenyl, alkynyl, etc. denote both straight and branched groups, but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A candidate molecule or compound described herein may be in an amount in a formulation or medicament, which is an amount that can lead to a biological effect, or lead to ameliorating, alleviating, lessening, relieving, diminishing or removing symptoms of a condition, e.g., disease, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism (microbe) in a system (e.g., cell, tissue, or subject) infected with a microbe, reducing the rate of microbial propagation, reducing the number of symptoms or an effect of a symptom associated with the microbial infection, and/or removing detectable amounts of the microbe from the system. Examples of microbe include but are not limited to virus, bacterium and fungus.

The term “therapeutically effective amount” as used herein refers to an amount of a compound, or an amount of a combination of compounds, to treat or prevent a disease or disorder, or to treat a symptom of the disease or disorder, in a subject. As used herein, the terms “subject” and “patient” generally refers to an individual who will receive or who has received treatment (e.g., administration of a compound) according to a method described herein.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present disclosure.

The terms “subject,” “patient” or “subject in need thereof” refers to a living organism suffering from or prone to a disease or condition that can be treated by administration of a compound, pharmaceutical composition, mixture or vaccine as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

The term “effective amount” as used herein refers to an amount effective to achieve an intended purpose. Accordingly, the terms “therapeutically effective amount” and the like refer to an amount of a compound, mixture or vaccine, or an amount of a combination thereof, to treat or prevent a disease or disorder, or to treat a symptom of the disease or disorder, in a subject in need thereof.

### Overview

Herein it is shown that GAPs are controlled by intestinal IL-6 signal transducer (IL6ST/gp130). Although mice that express constitutively active gp130/IL-6 signal transducer (IL6ST) in intestinal epithelial cells (IEC; gp130^{Act/IEC} mice) have fewer GCs (Taniguchi et al., 2015), they are ALD resistant due to increased GAP opening or formation, which enhances the generation of tolerogenic LP-APCs and production of IL-22 by type-3 innate lymphoid cells (ILC3). GAP opening induced an intestinal C-type regenerating islet derived-3 (Reg3) lectin-mediated antibacterial defense, reducing bacterial translocation to the liver and preventing alcoholic steatohepatitis. gp130 activation exerted its protective effects via muscarinic acetylcholine (ACh) receptor 4 (mAChR4), whose GC expression was induced by IL-6. Based on these findings, we developed a new therapeutic approach, administering a mAChR4 positive allosteric modulator (PAM) to stimulate intestinal GAP formation, thereby increasing tolerogenic LP-APCs and Reg3 expression, which prevented microbial translocation and protected mice from alcoholic steatohepatitis. The results show that IL6ST signaling modulates intestinal immunity through mAChR4. GAP induction by mAChR4 PAMs is a strategy for enhancing intestinal immune tolerance and interception with ALD and other diseases such as those linked to uncontrolled microbial translocation.

### Exemplary Compounds

The chemical genera provided herein are intended to be understood as describing “chemically feasible” structures, by which is meant that the structure depicted by any combination or subcombination of optional substituents meant to be recited by the claim is physically capable of existence with at least some stability as can be determined by the laws of structural chemistry and by experimentation. Structures that are not chemically feasible are not within a claimed set of compounds.

The term “alkyl” as used herein refers to substituted or unsubstituted straight chain, branched, saturated hydrocarbon group. The group can have from 1 to 10 carbon atoms, 1 to 6 carbon atoms, 1 to 4 carbon atoms, 2 to 6 carbon atoms, or 2 to 4 carbon atoms. Examples of straight chain alkyl groups include methyl (i.e., CH3), ethyl, n-propyl, n-butyl, n-pentyl, n-hexyl, n-heptyl, n-octyl groups. Examples of branched alkyl include isopropyl, iso-butyl, sec-butyl, t-butyl, neopentyl, and isopentyl. An alkyl can be optionally substituted.

The term “cycloalkyl” as used herein refers to substituted or unsubstituted cyclic hydrocarbon group, which may be saturated or partially saturated. The group can have from 3 to 10 carbon atoms, 3 to 8 carbon atoms, or 3 to 6 carbon atoms, 3 to 6 carbon atoms, or 2 to 4 carbon atoms. Examples of cycloalkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups. When optionally substituted, alkyl includes alkyl can be trifluoromethyl, difluoromethyl, or fluoromethyl. A cycloalkyl can be optionally substituted.

The term “aryl” as used herein refers to a cyclic aromatic hydrocarbon group. The group can have from 6 to about 10 carbon atoms, 10 to 20 carbon atoms, or about 6 carbon atoms. Examples include phenyl and naphthyl. An aryl can be optionally substituted.

The term “heterocyclyl” or “heterocycloalkyl” as used herein refers to non-aromatic heterocyclic group. The group may be saturated or partially saturated. The group can have from a ring size of 3 to 10 atoms, 4 to 7 atoms, or 5 to 6 atoms. For example, the ring can have 1-5 carbon atoms and 1 nitrogen atom. Examples of heterocycloalkyl groups include piperazine, piperidine, dioxolane, dioxane, pyrrolidine, tetrahydrothiophene, tetrahydrofuran, dihydrothiophene, or dihydrofuran. A heterocyclyl can be optionally substituted.

The term “heteroaryl” or “hetaryl” as used herein refers to an aromatic heterocyclic group. The group can have from a ring size of 5 to 10 atoms, 5 to 9 atoms, or 5 to 6 atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzirnidazolyl, azabenzirnidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl. A heteroaryl can be optionally substituted.

In general, “substituted” and “substituent” refers to an organic group as defined herein in which one or more bonds to a hydrogen atom contained therein are replaced by one or more bonds to a non-hydrogen atom such as, but not limited to, a halogen (i.e., “halo” selected from F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboyxlate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)2, CN, CF3, OCF3, R′, O, S, C(O), S(O), methylenedioxy, ethylenedioxy, N(R′)2SR′, SOR′, SO2R′, SO1N(R′)2, SO3R′, C(O)R′, C(O)C(O)R′, C(O)CH2C(O)R′, C(S)R′, C(O)OR′, OC(O)R′, C(O)N(R′)2, OC(O)N(R′)2, C(S)N(R′)2, (CH2)O-2NHC(O)R′, (CH2)0-2N(R′)N(R′)2, N(R′)N(R′)C(O)R′, N(R′)N(R′)C(O)OR′, N(R′)N(R′)CON(R′)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety, and wherein the carbon-based moiety can itself be further substituted. For example, R′ group can be hydrogen, C1-C6 alkyl, or phenyl.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH4+ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present disclosure may take the form of salts. The term “salts” embraces addition salts of free acids or free bases which are compounds. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds.

Suitable pharmaceutically-acceptable acid addition salts may be prepared from an inorganic acid or from an organic acid. Examples of inorganic acids include hydrochloric, hydrobrornic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula I or II compounds, for example in their purification by recrystallization. All of these salts may be prepared by conventional means from the corresponding compound. according to Formula I or II by reacting, for example, the appropriate acid or base with the compound according to Formula I or II. The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

The term “prodrug” means a derivative of a compound that can hydrolyze, oxidize, or otherwise react under biological conditions (in vitro or in vivo) to provide an active compound, particularly a compound disclosed herein. Examples of prodrugs include, but are not limited to, derivatives and metabolites of a compound disclosed herein that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

In one embodiment, the modulator has a structure according to Formula I, or a pharmaceutically acceptable salt thereof:

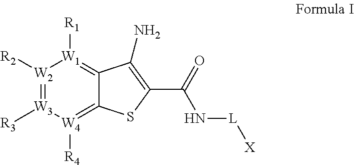

wherein
each of w₁, w₂, w₃, and w₄ is independently C or N;
each of R₁, R₂, R₃, and R₄ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH₂-heterocycloalkyl, —CH₂-aryl, or —CH₂-heteroaryl, CHF₂, CH₂F, or CF₃;
L is absent or CH₂;
X is a cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula I, or a pharmaceutically acceptable salt thereof:

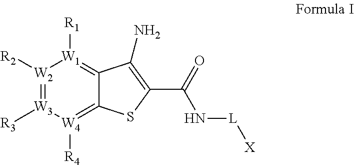

wherein
each of w₁, w₂, w₃, and w₄ is independently C or N;
each of R₁, R₂, R₃, and R₄ is independently H, F, Cl, Br, alkyl, ORₐ, or a lone pair if the group to which it is attached is N;
Rₐ is H, alkyl, or —CH₂-pyridine;
L is absent or CH₂;
X is a phenyl, pyridine or cyclopropyl, each of which may be optionally substituted with one or more group selected from Y, or two Y are linked so as to form a fused 1,3-dioxolane together with the atoms to which they are attached; and
Y is independently F, Cl, Br, CHF₂, CH₂F, CF₃, SO2R_{b}, SOR_{b}, SO, SR_{b}, SF₅, phenyl optionally substituted by Z, pyridine optionally substituted by Z, pyrimidine optionally substituted by Z, pyridazine optionally substituted by Z, pyrazine optionally substituted by Z,
Z is methyl, F, or OMe; and
R_{b} is H, alkyl, cycloalkyl, CHF₂, CH₂F, or CF₃.

In one embodiment, w₁ and w₂ are each C and w₃ and w₄ are each N. In one embodiment, w₁ and w₂ are each C and w₃ and w₄ are each N. In one embodiment, w₁, w₂, and w₃ are each C, and w₄ is N. In one embodiment, w₁, w₂, and w₃ are each C, and w₄ is N.

In one embodiment, the modulator has a structure according to Formula II, or a pharmaceutically acceptable salt thereof:

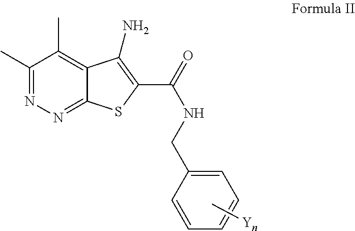

wherein
Y is independently F, Cl, Br, CHF₂, CH₂F, CF₃, SO2R_{b}, SOR_{b}, SO, SR_{b}, SF₅, phenyl optionally substituted by Z, pyridine optionally substituted by Z, pyrimidine optionally substituted by Z, pyridazine optionally substituted by Z, pyrazine optionally substituted by Z,
Z is methyl, F, or OMe;
R_{b} is H, alkyl, cycloalkyl, CHF₂, CH₂F, or CF₃; and
wherein n is 0-3.

In one embodiment, the modulator has a structure according to Formula III, or a pharmaceutically acceptable salt thereof:

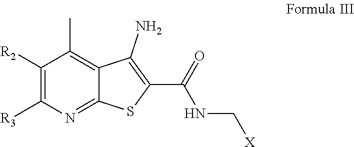

wherein
each of R₂, R₃, and R₄ is independently H, F, Cl, alkyl, ORₐ;
Rₐ is alkyl, or —CH₂-pyridine;
X is a phenyl or pyridine, each of which may be optionally substituted with one or more group selected from Y, or two Y are linked so as to form a fused 1,3-dioxolane together with the atoms to which they are attached; and
Y is independently F, Cl, Br, CHF₂, CH₂F, CF₃, OR_{b}, SO2R_{b}, SOR_{b}, SO, SR_{b}, SF₅, phenyl optionally substituted by Z, pyridine optionally substituted by Z, pyrimidine optionally substituted by Z, pyridazine optionally substituted by Z, pyrazine optionally substituted by Z,
Z is methyl, F, or OMe; and
R_{b} is H, alkyl, cycloalkyl, CHF₂, CH₂F, or CF₃.

In one embodiment, the modulator has a structure according to Formula IV, or a pharmaceutically acceptable salt thereof:

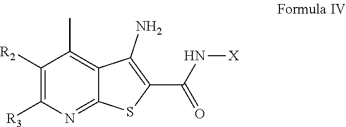

wherein each of R₂, R₃, and R₄ is independently H, F, Cl, or alkyl; and X is alkyl, cycloalkyl, aryl, heterocycloalkyl, or heteroaryl.

In one embodiment, the modulator has a structure according to Formula V, or a pharmaceutically acceptable salt thereof:

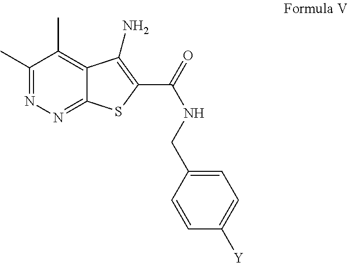

wherein Y is independently F, Cl, Br, CHF₂, CH₂F, CF₃, SO2R_{b}, SOR_{b}, SO, SR_{b}, SF₅; and R_{b} is H, alkyl, cycloalkyl, CHF₂, CH₂F, or CF₃.

In one embodiment, the modulator has a structure according to Formula VI, or a pharmaceutically acceptable salt thereof:

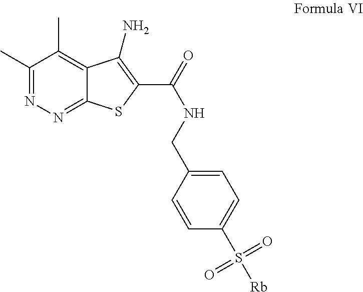

wherein R_{b} is H, alkyl, cycloalkyl, CHF₂, CH₂F, or CF₃.

In one embodiment, the modulator has the structure:

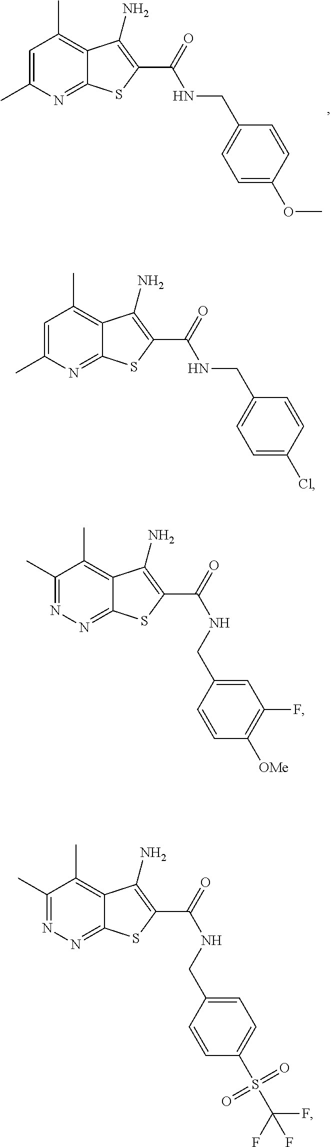

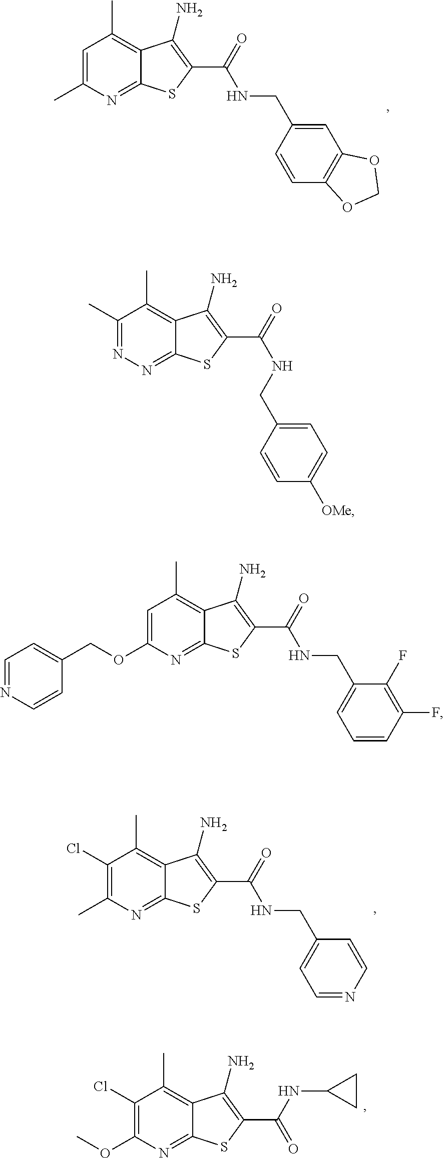 [SMILES: Cc1cc(C)c2c(N)c(C(=O)NCc3ccc4c(c3)OCO4)sc2n1 ; COc1ccc(CNC(=O)c2sc3nnc(C)c(C)c3c2N)cc1 ; Cc1cc(OCc2ccncc2)nc2sc(C(=O)NCc3cccc(F)c3F)c(N)c12 ; Cc1nc2sc(C(=O)NCc3ccncc3)c(N)c2c(C)c1Cl ; COc1nc2sc(C(=O)NC3CC3)c(N)c2c(C)c1Cl]

or a pharmaceutically acceptable salt thereof.

In one embodiment, the modulator has the structure: VU0467485, ML173, VU0448088, VU0464090, or a pharmaceutically acceptable salt thereof.

In one embodiment, the modulator has the structure: VU0467154, VU0152100, VU0152099, LY2033298, or VU010010, or a pharmaceutically acceptable salt thereof.

In one embodiment, the modulator has the structure:

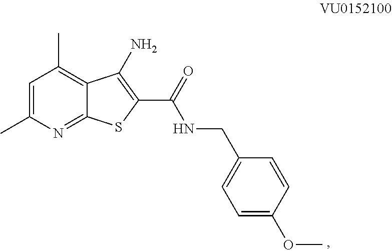

or a prodrug or pharmaceutically acceptable salt thereof.

In one embodiment, the modulator has the structure:

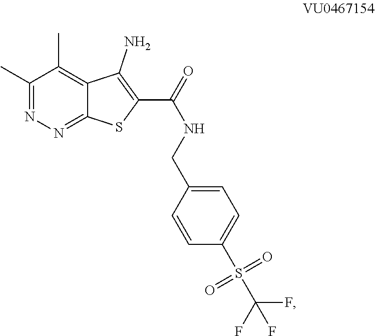 [SMILES: Cc1nnc2sc(C(=O)NCc3ccc(S(=O)(=O)C(F)(F)F)cc3)c(N)c2c1C]

or a prodrug or pharmaceutically acceptable salt thereof.

In one embodiment, the modulator has the structure:

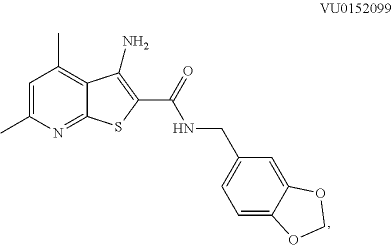 [SMILES: Cc1cc(C)c2c(N)c(C(=O)NCc3ccc4c(c3)OCO4)sc2n1]

or a prodrug or pharmaceutically acceptable salt thereof.

In one embodiment, the modulator has the structure:

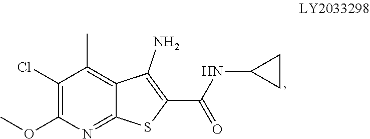 [SMILES: COc1nc2sc(C(=O)NC3CC3)c(N)c2c(C)c1Cl]

or a prodrug or pharmaceutically acceptable salt thereof.

In one embodiment, the modulator has the structure:

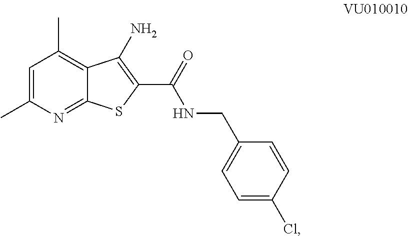 [SMILES: Cc1cc(C)c2c(N)c(C(=O)NCc3ccc(Cl)cc3)sc2n1]

or a prodrug or pharmaceutically acceptable salt thereof.

In one embodiment, the modulator has the structure:

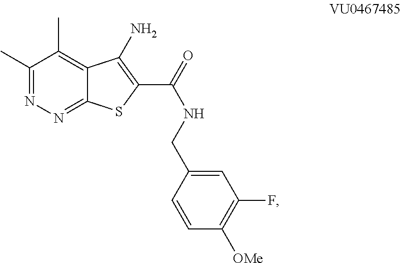 [SMILES: COc1ccc(CNC(=O)c2sc3nnc(C)c(C)c3c2N)cc1F]

or a prodrug or pharmaceutically acceptable salt thereof.

In one embodiment, the modulator has the structure:

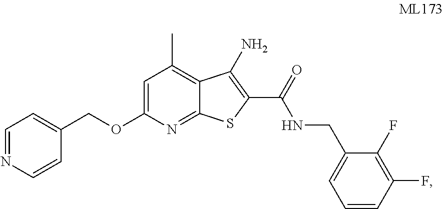 [SMILES: Cc1cc(OCc2ccncc2)nc2sc(C(=O)NCc3cccc(F)c3F)c(N)c12]

or a prodrug or pharmaceutically acceptable salt thereof.

In one embodiment, the modulator has the structure:

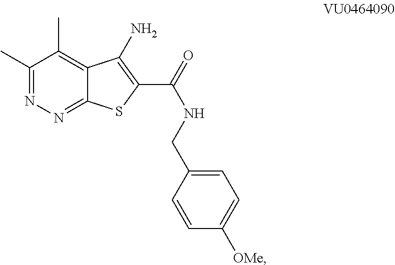 [SMILES: COc1ccc(CNC(=O)c2sc3nnc(C)c(C)c3c2N)cc1]

or a prodrug or pharmaceutically acceptable salt thereof.

In one embodiment, the modulator has the structure:

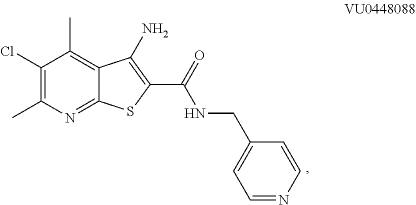 [SMILES: Cc1nc2sc(C(=O)NCc3ccncc3)c(N)c2c(C)c1Cl]

or a prodrug or pharmaceutically acceptable salt thereof.

In one embodiment, the modulator has the structure:

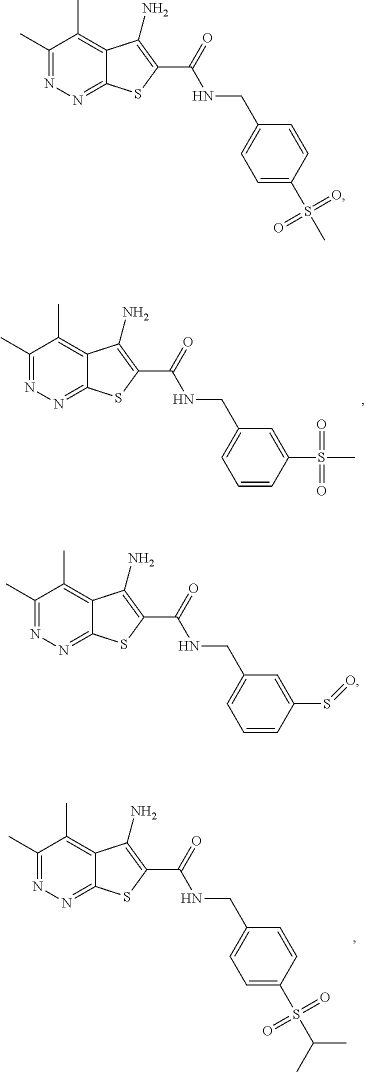

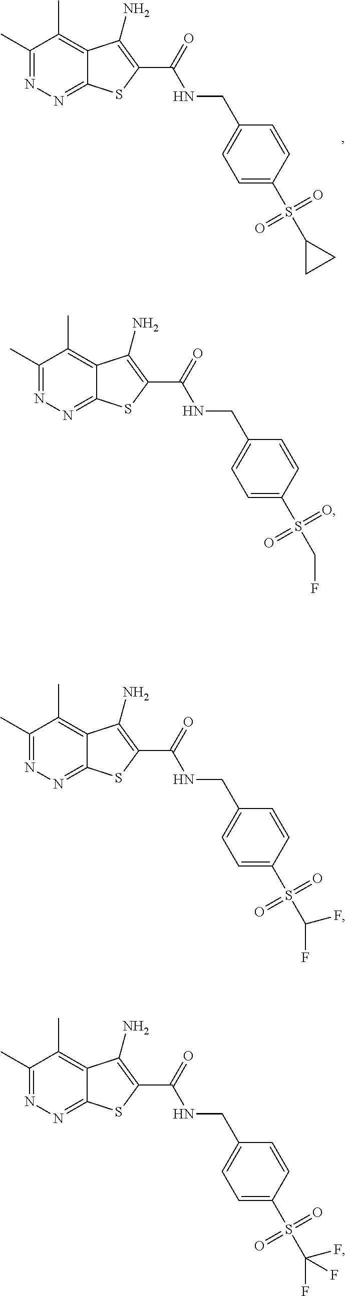 [SMILES: Cc1nnc2sc(C(=O)NCc3ccc(S(=O)(=O)C4CC4)cc3)c(N)c2c1C ; Cc1nnc2sc(C(=O)NCc3ccc(S(=O)(=O)CF)cc3)c(N)c2c1C ; Cc1nnc2sc(C(=O)NCc3ccc(S(=O)(=O)C(F)F)cc3)c(N)c2c1C ; Cc1nnc2sc(C(=O)NCc3ccc(S(=O)(=O)C(F)(F)F)cc3)c(N)c2c1C]

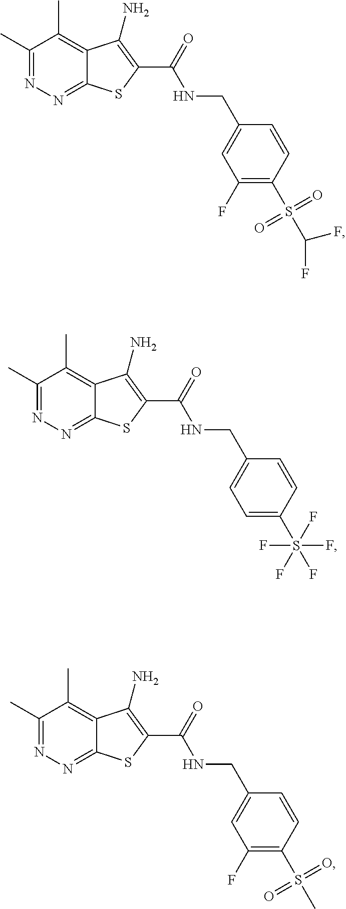 [SMILES: Cc1nnc2sc(C(=O)NCc3ccc(S(=O)(=O)C(F)F)c(F)c3)c(N)c2c1C ; Cc1nnc2sc(C(=O)NCc3ccc(S(F)(F)(F)(F)F)cc3)c(N)c2c1C ; Cc1nnc2sc(C(=O)NCc3ccc(S(C)(=O)=O)c(F)c3)c(N)c2c1C]

or a pharmaceutically acceptable salt thereof.

In one embodiment, the modulator has a structure according to Formula VII, or a pharmaceutically acceptable salt thereof:

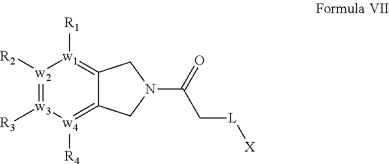

wherein
each of w₁, w₂, w₃, and w₄ is independently C or N;
each of R₁, R₂, R₃, and R₄ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
L is absent or CH₂;
X is a cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula VIII, or a pharmaceutically acceptable salt thereof:

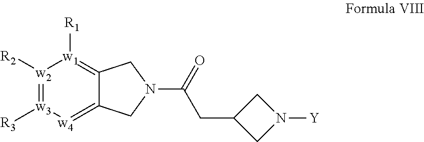

each of w₁, w₂, and w₃, is independently C or N;
each of R₁, R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula IX, or a pharmaceutically acceptable salt thereof:

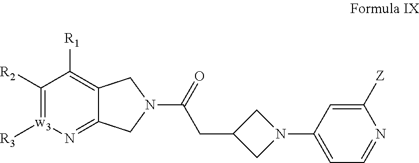

each of w₁, w₂, and w₃, is independently C or N;
each of R₁, R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula Xa, or a pharmaceutically acceptable salt thereof:

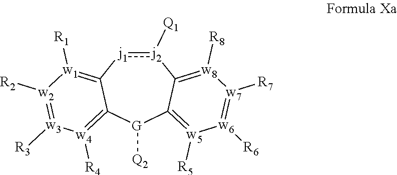

wherein
each of w₁, w₂, w₃, w₄, w₅, w₆, w₇, and w₈, is independently C or N;
each of R₁, R₂, R₃, R₄, R₅, R₆, R₇, and R₈ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
each of j₁, j₂, and G is independently CH, CH₂, N, O, S, SO2, SO, or NRₐ;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
one of Q₁ and Q₂ is X and the other is H, or a lone pair if the group to which it is attached is N, O, or S;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached, and when X is attached to G the bond between X and G is a single bond or a double bond;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula Xb, or a pharmaceutically acceptable salt thereof:

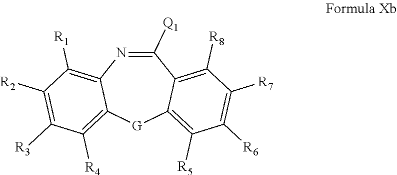

wherein
each of R₁, R₂, R₃, R₄, R₅, R₆, R₇, and R₈ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro;
G is independently CH₂, O, S, SO2, SO, CRₐ, or NRₐ;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q₁ is X;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached, and when X is attached to G the bond between X and G is a single bond or a double bond;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula XI, or a pharmaceutically acceptable salt thereof:

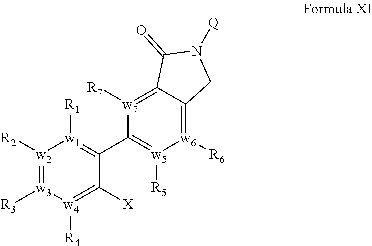

wherein
each of w₁, w₂, w₃, w₄, w₅, w₆, and w₇, is independently C or N;
each of R₁, R₂, R₃, R₄, R₅, R₆, and R₇, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q is H, alkyl, cycloalkyl, heterocycloalkyl, aryl, or heteroaryl, each of which may be optionally radiolabeled;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula XII, or a pharmaceutically acceptable salt thereof:

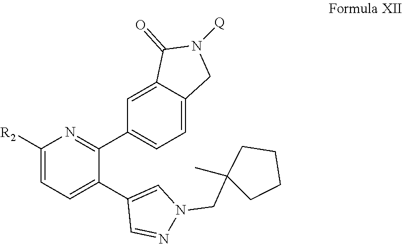

wherein
each R₂ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q is H, alkyl, cycloalkyl, heterocycloalkyl, aryl, or heteroaryl, each of which may be optionally radiolabeled.

In one embodiment, the modulator has a structure according to Formula XIII, or a pharmaceutically acceptable salt thereof:

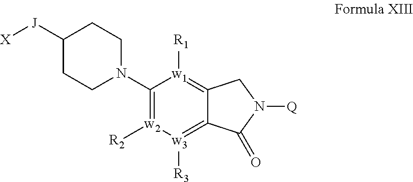

wherein
each of w₁, w₂, and w₃, is independently C or N;
each of R₁, R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q is H, alkyl, cycloalkyl, heterocycloalkyl, aryl, or heteroaryl, each of which may be optionally radiolabeled;
J is CHRₐ, O, S, NRₐ, or absent;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula XIV, or a pharmaceutically acceptable salt thereof:

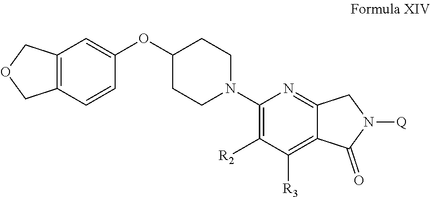

wherein
each of R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃; and
Q is H, alkyl, cycloalkyl, heterocycloalkyl, aryl, or heteroaryl, each of which may be optionally radiolabeled.

In one embodiment, the modulator has a structure according to Formula XV, or a pharmaceutically acceptable salt thereof:

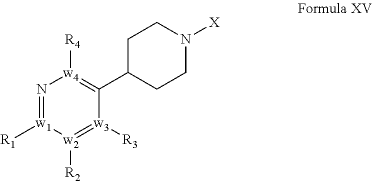

wherein
each of w₁, w₂, w₃, and w₄, is independently C or N;
each of R₁, R₂, R₃, and R₄, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
X is cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula XVI, or a pharmaceutically acceptable salt thereof:

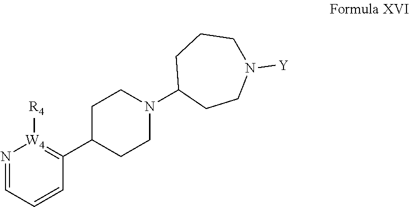

wherein
w₄ is independently C or N;
R₄ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z; and
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and R_{b} is H, alkyl, cycloalkyl, CHF₂, CH₂F, or CF₃.

In one embodiment, the modulator has a structure according to Formula XVII, or a pharmaceutically acceptable salt thereof:

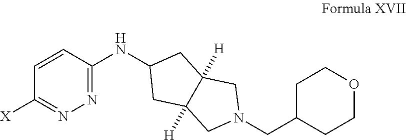

wherein
X is cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

In one embodiment, the modulator has a structure according to Formula XVIII, or a pharmaceutically acceptable salt thereof:

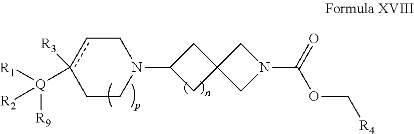

wherein
n is 1 or 2
p is 0, 1 or 2
Q is a five, six or seven membered monocyclic heterocyclic ring containing 1, 2, 3 or 4 heteroatom ring members selected from N, O and S;
R1 is selected from hydrogen; fluorine; chlorine; bromine; cyano; oxo; hydroxy; OR5; NR5R6; COR5; COOR5; OCOR5; NR7COR5; CONR5R6; NR7CONR5R6; NR7000R5; OCONR5R6; SR5; SOR5 and S02R5; a Ci.e non-aromatic hydrocarbon group which is optionally substituted with one to six fluorine atoms and wherein one or two, but not all, carbon atoms of the hydrocarbon group may optionally be replaced by a heteroatom selected from O, N and S and oxidized forms thereof; and an optionally substituted 5- or 6-membered ring containing 0, 1, 2 or 3 heteroatoms selected from O, N and S and oxidized forms thereof;
R2 is selected from hydrogen; fluorine; chlorine; bromine; cyano; hydroxy; methoxy; OR5; NR5R6; COR5; COOR5; OCOR5; NR7COR5; CONR5R6;
NR7CONR5R6; NR7000R5; OCONR5R6; SR5; SOR5 and S02R5; and a d.6 non-aromatic hydrocarbon group; or R1 and R2 can be joined together to form a 6 membered fused aromatic ring;
R9 is selected from hydrogen, CH3, CH2OH, CH(CH3)OH, C(CH3)2OH and COOCH3;
R3 is selected from hydrogen; fluorine; cyano; hydroxy; amino; and a Ci_9 non-aromatic hydrocarbon group which is optionally substituted with one to six fluorine atoms and wherein one, two or three, but not all, carbon atoms of the hydrocarbon group may optionally be replaced by a heteroatom selected from O, N and S and oxidized forms thereof; R4 is a hydrogen or a Ci_6 non-aromatic hydrocarbon group which is optionally substituted with one to six fluorine atoms and wherein one or two, but not all, carbon atoms of the hydrocarbon group may optionally be replaced by a heteroatom selected from O, N and S and oxidised forms thereof;
R5, R6 and R7 are the same or different and each is independently selected from hydrogen, a non-aromatic Ci_4 hydrocarbon group optionally substituted with one or more fluorine atoms, or a group of formula CH2N(Rₐ)COORb;
Ra is selected from hydrogen and a non-aromatic Ci_4 hydrocarbon group; Rb is a non-aromatic Ci_4 hydrocarbon group which is optionally substituted with one or more groups selected from fluorine; chlorine; bromine; cyano; hydroxy; methoxy; amino; or a cycloalkyl, heterocycloalkyl, aryl or heteroaryl group; and the dotted line indicates an optional second carbon-carbon bond, provided that when a second carbon-carbon bond is present, then R3 is absent.

In one embodiment, the modulator has the structure:

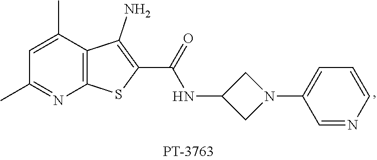 [SMILES: Cc1cc(C)c2c(N)c(C(=O)NC3CN(c4cccnc4)C3)sc2n1]

or a prodrug or pharmaceutically acceptable salt thereof.

In one embodiment the modulator has the structure:

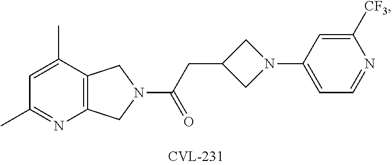 [SMILES: Cc1cc(C)c2c(n1)CN(C(=O)CC1CN(c3ccnc(C(F)(F)F)c3)C1)C2]

or a prodrug or pharmaceutically acceptable salt thereof.

In one embodiment, the modulator has the structure:

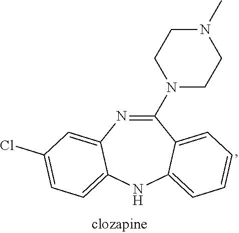 [SMILES: CN1CCN(C2=Nc3cc(Cl)ccc3Nc3ccccc32)CC1]

or a derivative, a prodrug, or pharmaceutically acceptable salt thereof.

In one embodiment, the modulator has the structure:

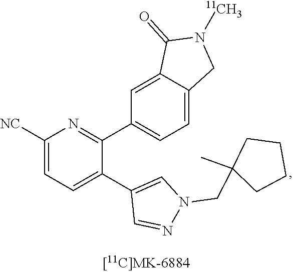

or a prodrug or pharmaceutically acceptable salt thereof.

In one embodiment, the modulator has the structure:

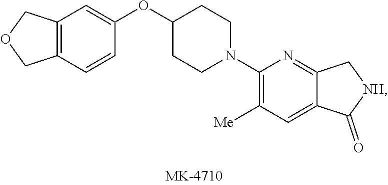 [SMILES: Cc1cc2c(nc1N1CCC(Oc3ccc4c(c3)COC4)CC1)CNC2=O]

or a prodrug or pharmaceutically acceptable salt thereof.

In one embodiment, the modulator has the structure:

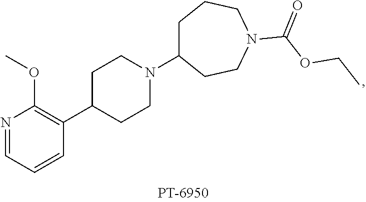 [SMILES: CCOC(=O)N1CCCC(N2CCC(c3cccnc3OC)CC2)CC1]

or a prodrug or pharmaceutically acceptable salt thereof.

In one embodiment, the modulator has the structure:

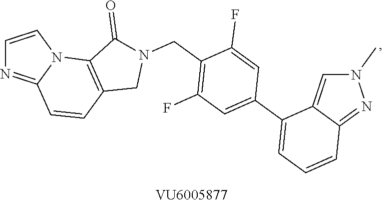 [SMILES: Cn1cc2c(-c3cc(F)c(CN4Cc5ccc6nccn6c5C4=O)c(F)c3)cccc2n1]

or a prodrug or pharmaceutically acceptable salt thereof.

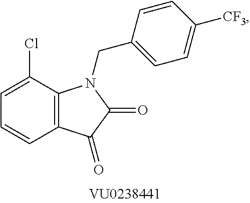 [SMILES: O=C1C(=O)N(Cc2ccc(C(F)(F)F)cc2)c2c(Cl)cccc21]

In one embodiment, the modulator has the structure:

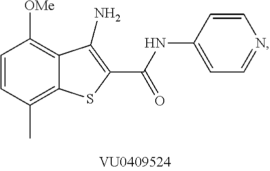 [SMILES: COc1ccc(C)c2sc(C(=O)Nc3ccncc3)c(N)c12]

or a prodrug or pharmaceutically acceptable salt thereof.

In one embodiment, the modulator has the structure:

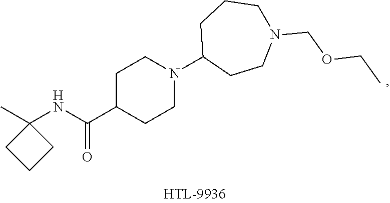 [SMILES: CCOCN1CCCC(N2CCC(C(=O)NC3(C)CCC3)CC2)CC1]

or a prodrug or pharmaceutically acceptable salt thereof.

In one embodiment, the modulator has the structure:

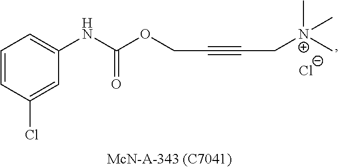 [SMILES: C[N+](C)(C)CC#CCOC(=O)Nc1cccc(Cl)c1 ; [Cl-]]

or a prodrug or pharmaceutically acceptable salt thereof.

In one embodiment, the modulator has the structure:

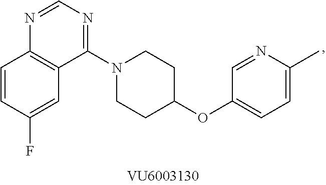 [SMILES: Cc1ccc(OC2CCN(c3ncnc4ccc(F)cc34)CC2)cn1]

or a prodrug or pharmaceutically acceptable salt thereof.

In one embodiment, the modulator has the structure:

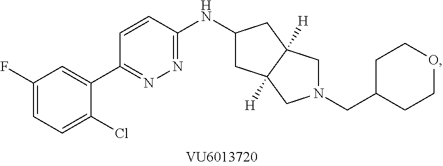 [SMILES: Fc1ccc(Cl)c(-c2ccc(NC3C[C@@H]4CN(CC5CCOCC5)C[C@@H]4C3)nn2)c1]

or a prodrug or pharmaceutically acceptable salt thereof.

In one embodiment, the modulator has the structure:

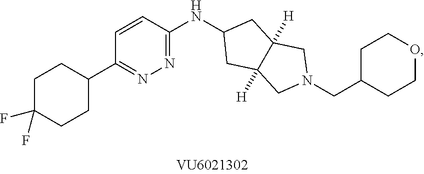 [SMILES: FC1(F)CCC(c2ccc(NC3C[C@@H]4CN(CC5CCOCC5)C[C@@H]4C3)nn2)CC1]

or a prodrug or pharmaceutically acceptable salt thereof.

In one embodiment, the modulator has the structure:

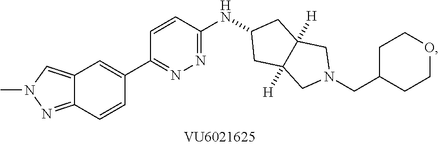 [SMILES: Cn1cc2cc(-c3ccc(N[C@H]4C[C@@H]5CN(CC6CCOCC6)C[C@@H]5C4)nn3)ccc2n1]

or a prodrug or pharmaceutically acceptable salt thereof.

In one embodiment, the modulator has the structure:

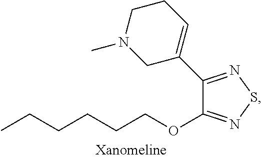 [SMILES: CCCCCCOc1nsnc1C1=CCCN(C)C1]

or a prodrug or pharmaceutically acceptable salt thereof.

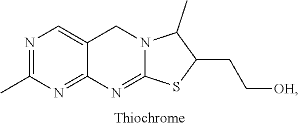 [SMILES: Cc1ncc2c(n1)N=C1SC(CCO)C(C)N1C2]

In one embodiment, the modulator has the structure:

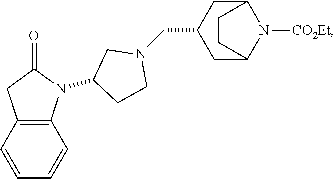

or a prodrug or pharmaceutically acceptable salt thereof.

In one embodiment, the modulator has the structure:

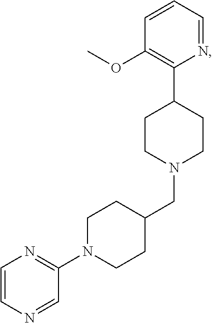 [SMILES: COc1cccnc1C1CCN(CC2CCN(c3cnccn3)CC2)CC1]

or a prodrug or pharmaceutically acceptable salt thereof.

In one embodiment, the modulator has the structure:

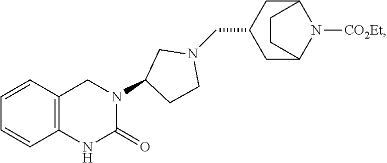

or a prodrug or pharmaceutically acceptable salt thereof.

In one embodiment, the modulator has the structure:

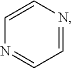

or a prodrug or pharmaceutically acceptable salt thereof.

In one embodiment, the modulator has the structure:

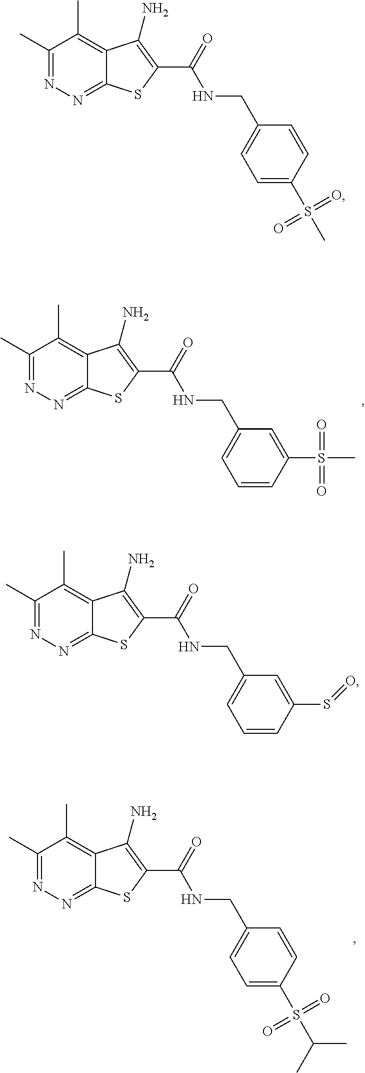

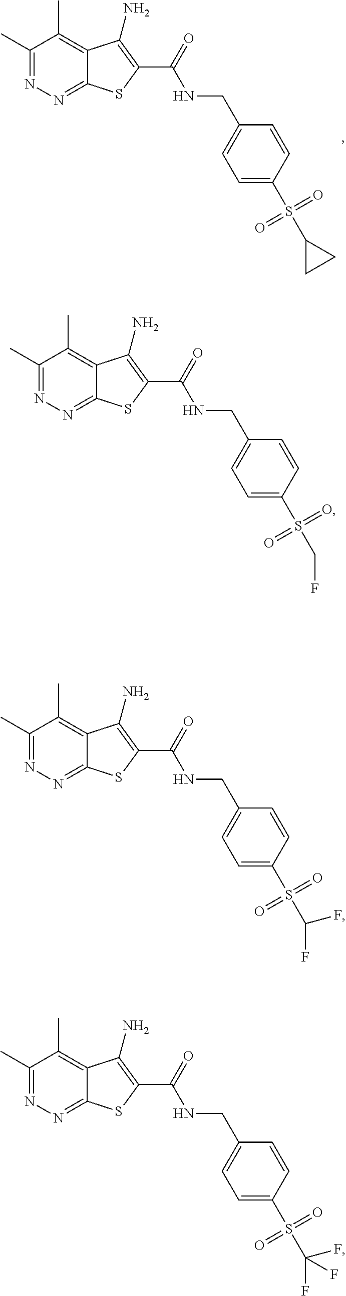 [SMILES: Cc1nnc2sc(C(=O)NCc3ccc(S(=O)(=O)C4CC4)cc3)c(N)c2c1C ; Cc1nnc2sc(C(=O)NCc3ccc(S(=O)(=O)CF)cc3)c(N)c2c1C ; Cc1nnc2sc(C(=O)NCc3ccc(S(=O)(=O)C(F)F)cc3)c(N)c2c1C ; Cc1nnc2sc(C(=O)NCc3ccc(S(=O)(=O)C(F)(F)F)cc3)c(N)c2c1C]

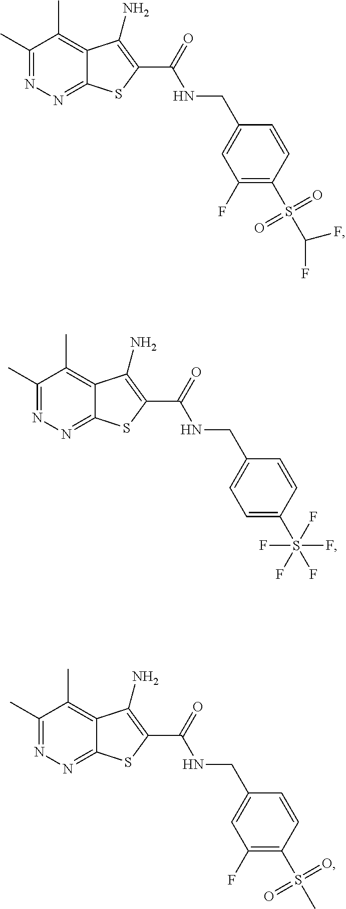 [SMILES: Cc1nnc2sc(C(=O)NCc3ccc(S(=O)(=O)C(F)F)c(F)c3)c(N)c2c1C ; Cc1nnc2sc(C(=O)NCc3ccc(S(F)(F)(F)(F)F)cc3)c(N)c2c1C ; Cc1nnc2sc(C(=O)NCc3ccc(S(C)(=O)=O)c(F)c3)c(N)c2c1C]

or a pharmaceutically acceptable salt thereof.

### Routes and Formulations

Administration of compositions having one or more AChR4 PAMs, optionally administered with a gp130 agonist, which may be administered concurrently with, before or after the AChR4 PAM, or any combination thereof, can be via any of suitable route of administration, particularly parenterally, for example, intravenously, intra-arterially, intraperitoneally, intrathecally, intraventricularly, intraurethrally, intrasternally, intracranially, intramuscularly, or subcutaneously. In one embodiment, the route is oral. In one embodiment, the route is intravenous. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds may be formulated as a sterile solution in water or another suitable solvent or mixture of solvents. The solution may contain other substances such as salts, sugars (particularly glucose or mannitol), to make the solution isotonic with blood, buffering agents such as acetic, critric, and/or phosphoric acids and their sodium salts, and preservatives.

In one embodiment, the compositions are administered orally, intravenously or intraperiotoneally. Sustained release formulations for longer duration of action may potenciate GAP opening, and/or more effective regulation of LP-DC.

The compositions alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the compositions alone or in combination with another active agent may be systemically administered, e.g., orally, in combination with a pharmaceutically acceptable vehicle such as an inert diluent or an assimilable edible carrier. For example, a AChR4 PAM may be administered with a gp130 agonsit such as IC7. They may be enclosed in hard or soft shell gelatin capsules, may be compressed into tablets, or may be incorporated directly with the food of the patient's diet. For oral therapeutic administration, the composition optionally in combination with another active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of the AChR4 PAM and optionally other active compounds in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The composition optionally in combination with another active compound may also be administered intravenously or intraperitoneally by infusion or injection. Solutions of AChR4 PAM in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the AChR4 PAM optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Excipients such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the disclosure provides various dosage formulations of the AChR4 PAM optionally in combination with another active compound for inhalation delivery. For example, formulations may be designed for aerosol use in devices such as metered-dose inhalers, dry powder inhalers and nebulizers.

Examples of useful dermatological compositions which can be used to deliver compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the active compound optionally in combination with another active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the AChR4 PAM optionally in combination with another active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 or 20 to 40 mg/kg/day or 15 mg to 60 mg or 20 to 40 mg per day. See, e.g.,

$$https : // www . clinicaltrials . gov / cts / show / NCT04787302 ? cond = muscarinic + acetylcholine + receptor + 4 \& draw = 2 \& rank = 1 ) .$$

The compound(s) optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 uM. In managing the patient, the therapy should be initiated at a lower dose and increased depending on the patient's global response.

The invention will be described by the following non-limiting example.

### EXAMPLE

### Methods

Human study. Duodenal biopsies from fifteen patients with AUD and eight biopsies from non-alcoholic controls were obtained from the alcohol withdrawal unit of Cliniques Universitaires Saint-Luc, Brussels, Belgium where they followed a highly standardized detoxification and rehabilitation program. The demographic data for controls and patients as well as clinical and laboratory characteristics for patients are shown in Table 1. AUD patients showed a wide range of liver involvement ranging from minimal liver disease to steatohepatitis or steatofibrosis but no patient had cirrhosis (Table 2). Patients were actively drinking until the day of admission. Exclusion criteria included antibiotics use during the two months preceding enrollment, immunosuppressive medication, diabetes, inflammatory bowel disease, known liver disease of any other etiology, and clinically significant cardio-vascular, pulmonary or renal co-morbidities. Written informed consent was obtained from all patients and controls. The study protocol was approved by the Ethics Committee of the Universite Catholique de Louvain, in Brussels, Belgium. Duodenal biopsies were obtained during an upper gastro-intestinal endoscopy performed at day 2 of admission. Samples were fixed in 10% formalin and were paraffin-embedded. To visualize GCs in humans, we used Periodic Acid/Schiff (PAS) staining. Slides were digitalized using a SCN400 slide scanner (Leica Biosystems, Wetzlar, Germany) at ×20 magnification and subjected to analysis with the image analysis tool Author version 2017.2 (Visiopharm, Horsolm, Denmark).

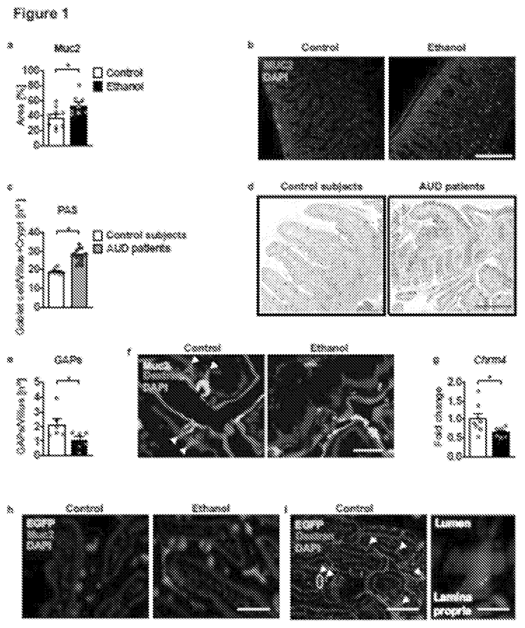

Mice. C57BL/6 mice were purchased from Charles River and used in FIG. 1. WT C57BL/6 mice bred at the UCSD animal facility were used in FIG. 4 and FIG. 9. All mice used in other figures were bred in the same animal facility. gp130^{Act/IEC} mice on a C57BL/6 background have been described before (Taniguchi et al., 2015100. gp130^{Act/IEC} males and WT littermate females were used for breeding. Littermates were used throughout this study except for ILC3 analysis where 6 non-littermate C57BL/6 WT were included in each group.

Female mice (age, 8 weeks) were placed on Lieber DeCarli diet for 10 weeks as previously described (Llorente et al., 2017). In brief, the Lieber DeCarli diet comprises Micro Stabilized Rod Liq AC IRR 438 (LD101A; TestDiet), Maltodextrin IRR (9598; TestDiet) and 200-proof ethanol (Koptec). The caloric intake from ethanol was 0 on day 1, 10% of total calories on days 2 and 3, 20% on days 4 and 5, 30% from day 6 until the end of 6 weeks, and 36% until the end of the treatment. Control mice received an isocaloric amount of iso-maltose instead of ethanol.

To study the effect of tropicamide, gp130^{Act/IEC} mice and corresponding WT littermate mice fed Lieber DeCarli diet containing ethanol or isocaloric iso-maltose for 10 weeks were treated with the mAChR4 antagonist tropicamide (20 mg/kg), which was dissolved in the diet for the last 29 days.

To enhance GAP formation, WT C57BL/6 mice fed Lieber DeCarli diet containing ethanol or isocaloric iso-maltose for 10 weeks were treated for the last 29 days with the specific mAChR4 PAM VU0467154 (5 mg/kg), which was dissolved in the liquid diet.

Mice were pair-fed and the amount of liquid diet containing ethanol was similar between mouse strains within each experiment (FIG. 5A and FIG. 5A). Activation of gp130 in IEC did not affect ethanol absorption (FIG. 5B). The use of tropicamide or VU0467154 did not affect ethanol absorption (FIG. 5B and FIG. 9B).

All animal studies were reviewed and approved by the UCSD Institutional Animal Care and Use Committee.

Bacterial DNA isolation and 16S rRNA sequencing. DNA was isolated from feces of mice. Samples were resuspended in phosphate-buffered saline (PBS) and digested with RNAse A and proteinase K at 55° C. for one hour. Each suspension was then transferred to individual Qbiogene lysing matrix B tubes and vortexed using a FastPrep FP120 instrument. The lysate was then extracted twice using Phenol/Chloroform/Isoamyl alcohol, precipitated and washed with ethanol, and the DNA resuspended in TE buffer (Yan et al., 2011; Fouts et al., 2012; Chen et al., 2015). 16S ribosomal RNA (rRNA) PCR was completed using Illumina adaptor and barcode ligated 16S primers targeting the V4 region of the 16S rRNA gene (Duan et al., Yan et al., 2011; Haas et al., 2011; Caporaso et al., 2011). Amplicons were purified using the Qiaquick PCR purification kit (QIAGEN) following manufacturer's specifications. Purified amplicons were then quantified via TECAN assay (Tecan, Switzerland), normalized, and pooled in preparation for 16S rRNA sequencing. The pooled library was quantified and checked for quality using Agilent 2100 Bioanalyzer (Agilent Technologies) and sequenced on Illumina MiSeq (Illumina) using V2 reagent chemistry, 500 cycles, 2×250 bp format using manufacturer's specifications. 16S sequence reads were processed and OTUs were determined using our MOTHUR-based 16S rDNA analysis workflow as described (Llorente et al., 2017; Yan et al., 2011; Chen et al., 2015). Raw 16S sequence reads can be found in the NCBI SRA associated with Bioproject PRJNA705611 and BioSample IDs: SAMN18094194-SAMN18094231.

Determination of bacterial translocation. Translocation of viable bacteria was assessed by culturing MLN and liver (Yan et al., 2011). Sterile MLN and liver were homogenized using a bead beater (1.0 mm zirconia/silica beads) under sterile and anaerobic conditions and kept for 1.5 hour at 37° C. in a bacterial incubator. Different dilutions were plated on CDC Anaerobe 5% Sheep Blood Agar with Phenylethyl Alcohol (PEA) plates inside of an anaerobic workstation and cultured anaerobically at 37° C. for 72 hours.

Staining procedures. Formalin-fixed tissue samples were embedded in paraffin (Paraplast plus, McCornick) and stained with H&E (Surgipath). To determine lipid accumulation, liver sections were embedded in OCT (Tissue-Tek^{R}) compound. 5 μm frozen sections were then cut and stained with Oil Red O (Sigma-Aldrich). Representative pictures from each group of mice are shown in each figure. All samples were analyzed by densitometry, using National Institutes of Health (NIH) Image J.

For immunofluorescence staining, tissues were fixed in 10% buffered formalin, embedded in paraffin and sectioned at 5 μm thickness and stained with anti-Muc2 (1:200) (San Cruz Biotechnology) primary antibody, or anti-mAChR4 (1:200) (Alomone labs) overnight, or anti-GFP antibody (1:200) (Abcam) and then, incubated with Alexa fluor 568- or Alexa fluor 488-conjugated secondary antibodies (Invitrogen). Nuclei were stained in blue with a Vectashield^{R} (Vector Laboratories) mounting medium containing DAPI and imaged by fluorescent microscopy. Control sections were stained with isotype antibody and showed no staining. All samples were analyzed by densitometry, using NIH Image J.

To enumerate GAPs a 2 cm small intestinal loop was clipped and injected with 200 μl of 10 mg/ml tetramethylrhodamine (TMR)-dextran 10,000 MW, lysine fixable (Thermo Scientific). Next, tissue was fixed in 10% formalin overnight and subsequently embedded in OCT for frozen sectioning (5 μm) for Muc2 immunofluorescence staining as above. Number of GAPs were identified as TMR-dextran-filled columns measuring approximately 20 μm (height)×5 μm (diameter) traversing the epithelium and containing a nucleus. Small intestinal GAPs were enumerated as GAPs per villus. To evaluate the intercommunication of bacteria with LP-immune cells via GAPs, 5×10⁹ E. faecalis genetically modified with an EGFP vector were gavaged at 3 and 0.5 h before the end of the study. Trans-epithelial dextran columns did not cause disruption of the epithelial barrier as shown by the exclusion of dextran from the LP.

Bacterial culture. E. faecalis was genetically modified with an EGFP reporter plasmid pBSU101 (Aymanns et al., 2011). EGFP-E. faecalis were grown in brain heart infusion (BHI) broth or on BHI agar plate at 37° C. with 125 μg/ml spectinomycin (Sigma). 5×10⁹ CFUs were gavaged as indicated in figure legends.

Real-time quantitative PCR. RNA was extracted from mouse tissues and cDNA was generated 511 (Duan et al., 2019). Primer sequences for mouse genes were obtained from the NIH qPrimerDepot. All primers used in this study are listed in Table 3. Gene expression was determined with Sybr Green (Bio-Rad Laboratories) using ABI StepOnePlus real-time PCR system. The qPCR value was normalized to Tbp or 18S housekeeping genes. To quantify the total bacterial load in feces, the 515 qPCR value of 16S rRNA gene for each sample was multiplied by the total amount of DNA (μg) mg⁻¹ of feces. Published bacterial primer sequences were used for 16S rRNA gene (Maeda et al., 2003) (Table 3).

Biochemical analysis. Plasma levels of ALT were determined with Infinity ALT kit (Thermo Scientific). Hepatic triglyceride levels were measured using Triglyceride Liquid Reagents kit (Pointe Scientific). Plasma levels of ethanol were measured using Ethanol Assay kit (BioVision).

Goblet cell isolation. GCs were isolated by selection of biotinylated cytokeratin 18 (CK18) (Abcam) (Knoop et al., 2017b), which is highly expressed in GCs, with streptavidin magnetic beads after isolation of small intestinal cells using a solution containing HBSS without Ca2⁺ and Mg2⁺, 1M HEPES, 100 mM sodium pyruvate and 0.5 M EDTA. Magnitude of GC enrichment was confirmed (FIG. 5C-D).

Small intestinal organoid isolation and culture. Small intestinal crypts were isolated from WT mice, cultured and stained as described 41 and treated with ethanol (10 and 50 mM) and IL-6 (20 ng/ml) (Biolegend) for 12 hours.

Flow cytometry. Isolation: Small intestine was harvested, and the epithelial cell layer was removed. Pieces were digested in media containing 1 mg/mL collagenase (Millipore Sigma), 0.1 U/mL dispase (Worthington Biochem) and 0.1 mg/mL DNase I (Millipore Sigma) at 37° C. at 150 rpm for 30 minutes. A 40% and 80% Percoll gradient was used to collect the lymphoid fractions at the interphase. Flow cytometry: LP-immune cells were pelleted and blocked with anti-mouse CD16/32 antibody (Thermo Fisher) for 15 minutes. Cells were divided to perform 3 different panels to analyze APCs, ILC3 and Tregs.

Stimulation and staining: To stimulate IL-23 secretion by LP-DCs, cells were re-stimulated in vitro with 100 ng/ml flagellin (Invivogen) for 2.5 hours in the presence of 0.7 μl/ml BD GolgiStop™ (BD Bioscience) to accumulate cytokines and/or proteins. The murine APC panel consisted of CD45.2 (V500, clone 104, BD), CD11c (FITC, clone N418, ThermoFisher), MHC Class II (I-A/I-E) (PE, clone M5/114. 15.2, ThermoFisher), CD11b (PerCP544 Cy5.5, clone M1/70, BD), CD103 (APC-R700, clone M290, BD), CX3CR1 (BV421, clone SA011F11, BioLegend), fixable viability stain (FVS) (575V, BD), IL-10 (BV711, clone JES5-16E3, BD) and IL-23 p19 (AF647, clone N71-1183).

In parallel, another set of isolated LP-immune cells was stimulated with 40 ng/μl of recombinant mouse IL-23 (Biolegend) for 4 hours in the presence of 0.7 μl/ml BD GolgiStop™ (BD Bioscience) to stimulate IL-22 secretion. ILC3 consisted of CD45.2 (V500, clone 104, BD), CD3e (FITC, clone 17A2, PharMingen), FVS (575V, BD), IL-22 (APC, clone IL22JOP, ThermoFisher) and RORγt (PE, clone B2D, ThermoFisher).

Another set of isolated LP-immune cells were stimulated with 10 ng/ml phorbol 12-myristate 13-acetate (PMA) (Sigma-Aldrich) plus 500 ng/ml ionomycin (Sigma-Aldrich) for 4 hours in the presence of 0.7 μl/ml BD GolgiStop™ (BD Bioscience). The third panel consisted of FVS (575V, BD), CD45.2 (V500, clone 104, BD), CD4 (BUV496, clone GK1.5, BD), CD25 (APC-R700, clone PC61, BD), FOXP3 (BV421, clone MF-14, BioLegend).

Cells were stained with the corresponding antibodies against the surface receptors of interest for 30 minutes following permeabilization and intracellular staining of cytokines or receptors for 30 minutes. Cells were recorded using FACS Celesta and LSR Fortessa (BD) flow cytometers at the Flow Cytometry Core Facility at the La Jolla Institute for Immunology (La Jolla, CA). Data was analyzed using FlowJo (version 10.5.3).

Immunoblot analyses. To measure expression levels of mAChR4, GCs were isolated as described above. Immunoblot analysis was performed as described 33 using anti-AChR4 1:1000 (#AMR-004; Alomone labs) and anti-β-actin 1:5000 (Sigma-Aldrich) antibody as loading control. Protein from proximal small intestine was extracted and immunoblot analysis was performed using anti-Reg3g 1:500 (ab198216, Abcam), anti-Reg3b 1:500 (ABIN1870289, Antibodies online), and anti-IL-10 1:1000 (sc-365858; Santa Cruz Biotechnology) antibodies. Stain-free imaging technology using polyacrylamide gels (BioRad) containing a proprietary trihalo compound to make proteins fluorescent, was employed to calculate total protein. Immunoblots were visualized with a charged coupling device camera in a luminescent image analyzer (Gel-Doc; Bio-Rad). Immunoblots were analyzed by densitometry, using Image Lab version 6.0.1 software (Bio-Rad).

Data availability. Raw 16S sequence reads can be found in the NCBI SRA associated with Bioproject PRJNA705611 and BioSample IDs: SAMN18094194-SAMN18094231.

Statistical analysis. Results are expressed as mean±s.e.m. Significance of two groups or multiple groups were evaluated using two-sided unpaired Student's t-test, two-sided unpaired Mann-Whitney test, or one-way or two-way analysis of variance (ANOVA) with Tukey's post-hoc test, respectively. Statistical analyses were performed using R statistical software, R v.3.5.1 (R Foundation for Statistical Computing) and GraphPad Prism v9.01. A P<0.05 was considered to be statistically significant (adjusted for multiple comparison when performing multiple tests).

### Results

Alcohol consumption is the seventh leading risk factor for death worldwide (Collaborators, 2016), and ALD is the major cause of liver transplantation in the West (Lee et al., 2019). There is no treatment for ALD except for abstinence. Chronic alcohol abuse is associated with increased intestinal permeability, dysbiosis and translocation of viable bacteria, such as Enterococcus faecalis (E. faecalis), to the liver, enhancing inflammation and accelerating ALD progression (Llorente et al., 2017; Duan et al., 2019). Intestinal immune-microbiome interactions are altered during ALD (Bruellman & Llorente, 2017). Such findings suggest that the intestinal immune system may control ALD pathogenesis. To test this hypothesis and elucidate the underlying mechanisms we investigated how chronic alcohol exposure affects the intestinal immune system and bacterial translocation to the liver.

### Chronic Alcohol Abuse Alters Goblet Cell Biology in Mice and Humans

GCs secrete mucins which coat the IEC apical surface and prevent microbial adhesion and translocation (Velcich et al., 2002). Paradoxically, chronic ethanol administration increased production of mucin-2 (Muc2), the most abundant intestinal mucin (Hartmann et al., 2013), and the number of mucin secreting GCs in the small intestine of mice (FIGS. 1A-1B) and patients with alcohol use disorder (AUD) (FIGS. 1C-1D). In the small intestine, GCs deliver luminal antigens and bacteria to LP-APCs through GAPs, thereby inducing adaptive immune responses (McDole et al., 2012; Kulkarni et al., 2020). GAPs are formed immediately after mucin is secreted from the GC into the intestinal lumen (McDole et al., 2012). We observed that the number of GAPs, identified as dextran-filled columns traversing nucleated cells that are Muc2 positive, was reduced following chronic ethanol feeding (FIGS. 1E-1F). Small intestinal GAPs are dynamic and opening occurs in response to acetylcholine (ACh), which activates muscarinic acetylcholine receptor 4 (mAChR4) on GCs (Knoop et al., 2015; Denning et al., 2011). The observed reduction in GAP formation after ethanol exposure could be related to reduced expression of Chrm4, the gene for mAChR4 (FIG. 1G). When we gavaged mice with enhanced green fluorescent protein (EGFP)-E. faecalis, we found colocalization of bacteria with Muc2 inside GCs (FIG. 1H) and with dextran-filled GAP columns (FIG. 1I), suggesting that bacteria are sampled by GCs. These results indicate that chronic alcohol use increases protective mucin production, which paradoxically comes with the expense of GAP closure.

### IL6ST-Mediated Induction of GAPs Prevents Ethanol-Induced Liver Disease

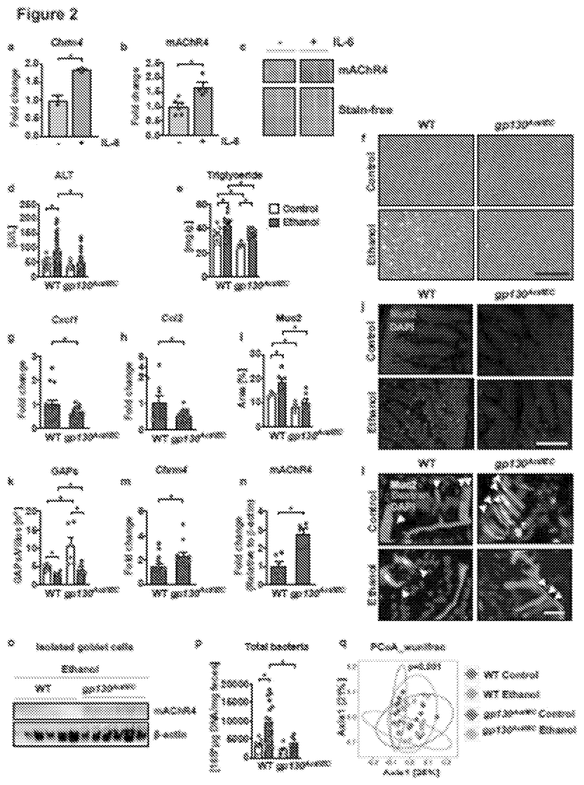

IL-6 is one of several cytokines elevated in ALD that affects the liver and the intestine (Hong et al., 2002). Although IL-6 correlates with liver disease severity (Sheron et al., 1991), it also exerts important barrier protective effects (Kuhn et al., 2018). Of note, IL-6 treatment induced mAChR4 expression in small intestinal organoids and cultured GCs (FIGS. 2A-2C). Previously, we showed that IL-6 signaling promotes intestinal stem cell survival and proliferation through activation of STAT3 and YAP and that gp130^{Act/IEC} mice are resistant to mucosal erosion, despite having fewer GCs than wild type (WT) mice (Taniguchi et al., 2015). Remarkably, gp130^{Act/IEC} mice were protected from ethanol-induced liver injury compared with WT littermates, as indicated by lower plasma alanine amino-transferase (ALT) (FIG. 2D), reduced hepatic steatosis (FIGS. 2E-2F) and reduced liver inflammation (FIGS. 2G-2H), while having fewer GCs and lower Muc2 expression in the small intestine (FIGS. 21-2J). Activation of gp130 in IEC did not affect food intake or intestinal ethanol absorption (FIGS. 5A-B). In accordance with the in vitro experiments, gp130^{Act/IEC} mice showed more GAPs in the small intestine (FIGS. 2K-2L), expressed higher amounts of Chrm4 mRNA (FIG. 2M) and mAChR4 protein in isolated GCs (FIGS. 2N-2O and FIGS. 5C-D). mAChR4 is expressed in IEC, GC, some LP immune cells and the enteric nervous system in the small intestine. mACR4 expression in GC was found distributed above the nuclei and subjacent to the secretory granules (5E). Ethanol-fed gp130^{Act/IEC} mice were also protected from intestinal bacterial overgrowth and showed a different fecal microbiota composition than WT littermates (FIGS. 2P-2Q). These results indicate that IL-6-gp130 regulation of intestinal GAPs impacts the development of ethanol-induced liver injury.

gp130^{Act/IEC} mice show elevated tolerogenic APCs and antimicrobial defense

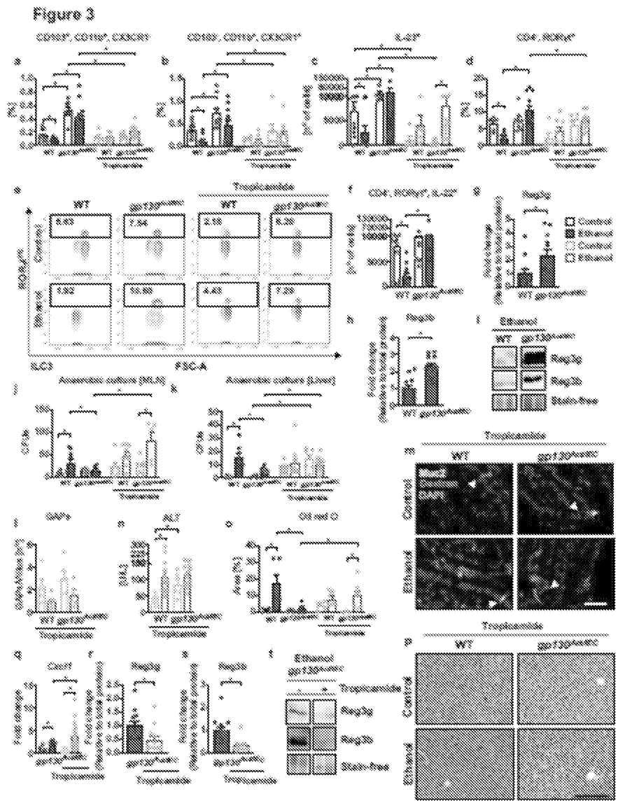

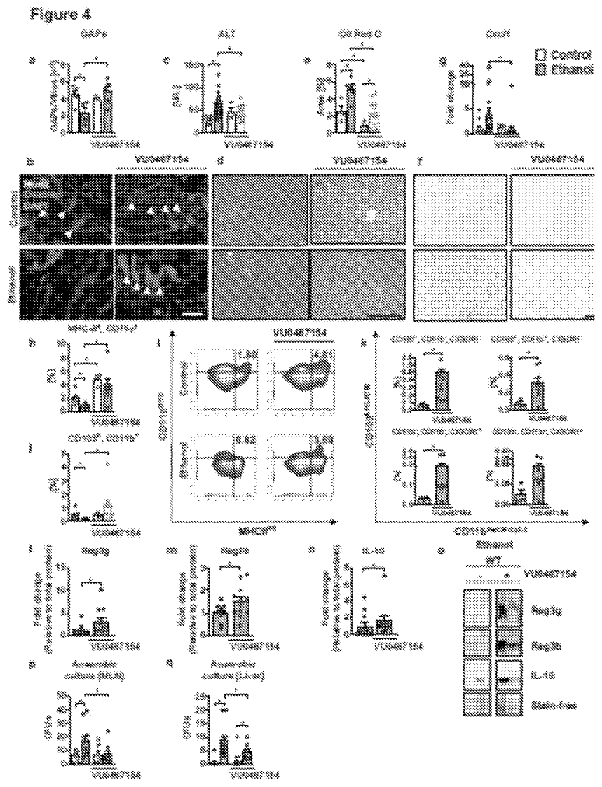

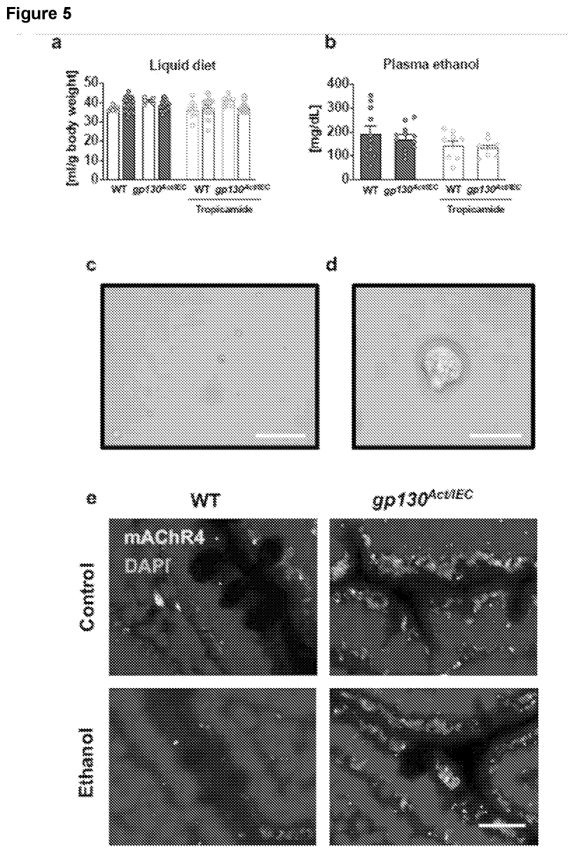

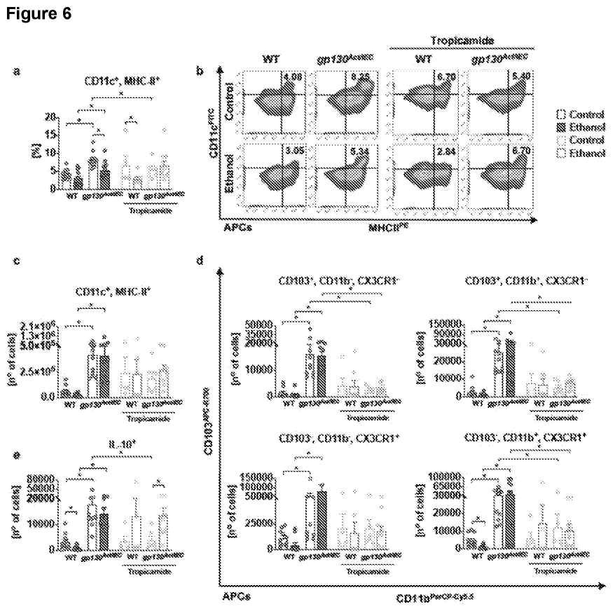

To study the consequences of GAP modulation, we characterized different subsets of CD11c⁺, major histocompatibility class (MHC)II⁺ LP-APCs (Denning et al., 2011). Chronic ethanol-feeding in WT mice did not affect frequencies or numbers of CD11c⁺, MHCII⁺ DCs (FIGS. 6A-C), but it reduced the frequencies of the CD103⁺, CD11b⁺, CX3CR1⁻ DC subset (FIG. 3A), frequencies and numbers of CD103⁻, CD11b⁺, CX3CR1⁺ APCs (FIGS. 3B and 6D), numbers of IL-23 expressing APC subsets (FIG. 3C), and IL-10 expressing APC subsets (FIG. 6E). Correspondingly, ethanol decreased frequencies of type-3 innate lymphoid cells (ILC3) (CD4⁻, RORγt⁺) and IL-22 expressing ILC3s (FIGS. 3D-3F).

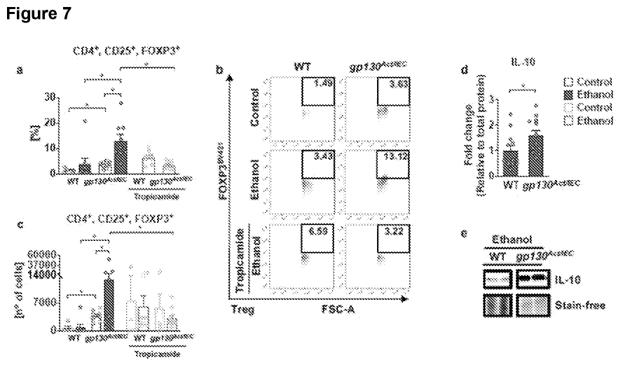

Alcohol-induced suppression of the intestinal immune system was restored in gp130^{Act/IEC} mice, which had increased frequencies and numbers of the tolerogenic APC subsets (CD103⁺, CD11b⁺, CX3CR1⁻ and CD103⁻, CD11b⁺, CX3CR1⁺) (FIGS. 3A-3B and FIG. 6D) and higher numbers of IL-23 and IL-10 expressing APC subsets (FIG. 3c and FIG. 6e). Frequencies of ILC3s (CD3⁻, ROR_{γ}t⁺) were also increased in ethanol-fed gp130^{Act/IEC} mice along with the number of IL-22 expressing ILC3s (FIGS. 3D-3F). As a consequence, the Reg3g and Reg3b antimicrobial peptides were upregulated in gp130^{Act/IEC} mice after ethanol administration (FIGS. 3G-3I). As we have reported, increased Reg3 lectins might not only contribute to protection from bacterial overgrowth (FIG. 2P) but can also inhibit bacterial translocation (Wang et al., 2016). Indeed, less viable bacteria translocated from the intestine to mesenteric lymph nodes (MLN) and liver of ethanol-fed gp130^{Act/IEC} mice relative to WT mice (FIGS. 3J-3K). Consistent with the increase in tolerogenic APC subsets, frequencies and numbers of Treg cells characterized as CD4⁺, CD25⁺, FOXP3⁺ and levels of intestinal IL-10 were elevated in gp130^{Act/IEC} mice after ethanol treatment (FIGS. 7a-3e). These results indicate that a specific tolerogenic APC-mediated intestinal adaptive immune response and antibacterial defenses might contribute to protection against ethanol-induced liver disease in mice.

gp130^{Act/IEC} mice lose protection from ethanol-induced liver disease on mAChR4 antagonism

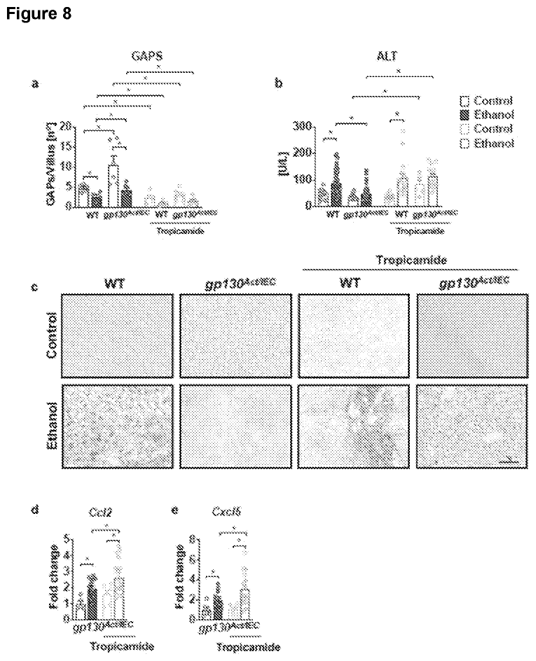

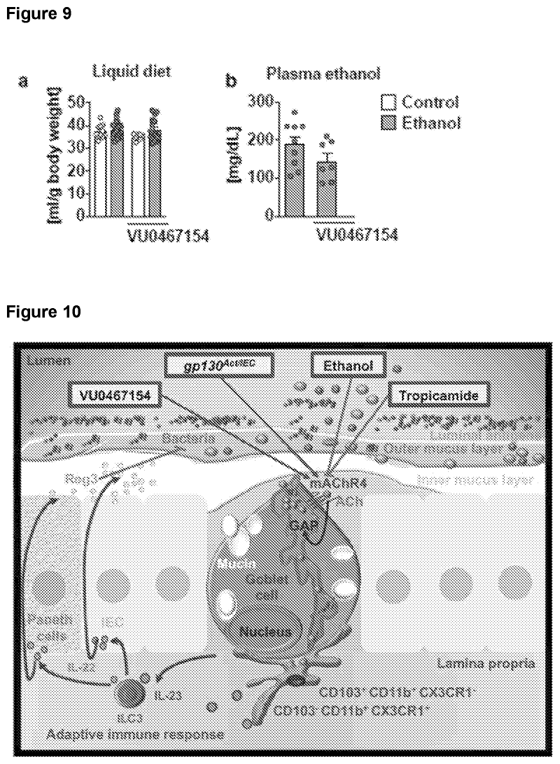

To determine whether gp130 exerts its protective effects via mAChR4-controlled GAP opening, mice were treated with the mAChR4 antagonist tropicamide. Tropicamide dissolved in the diet during the last 29 days of chronic ethanol feeding inhibited intestinal GAP formation (FIGS. 3L-3M and FIG. 8A) and made gp130^{Act/IEC} mice lose protection from liver disease (FIG. 4N and FIG. 8B), steatosis (FIGS. 3O-3P and FIG. 8C) and inflammation (FIG. 4Q, FIGS. 8D-4E). Despite a tendency, tropicamide did not worsen the disease in WT mice, probably because ethanol already inhibited GAP formation in these mice (FIGS. 1E-1G, FIG. 8A). Tropicamide did not affect food intake or intestinal ethanol absorption (FIGS. 5A-B). Tropicamide reverted the increase in GAP-associated CD103⁺, CD11b⁺, CX3CR1⁻ DCs and CD103⁻, CD11b⁺, CX3CR1⁺ APCs, ILC3s and Tregs in ethanol-fed gp130^{Act/IEC} mice (FIGS. 3A-3B, 3D-3F, FIG. 6D and FIGS. 7A-C). Tropicamide treatment also reversed Reg3b and Reg3g protein induction in ethanol-fed gp130^{Act/IEC} mice (FIGS. 3R-3T), and significantly increased bacterial translocation to MLN and liver in ethanol-fed gp130^{Act/IEC} mice (FIGS. 3J-3K). Tropicamide treatment did not affect food intake or intestinal ethanol absorption (FIGS. 5A-B). These data demonstrate that intestinal gp130 activation exerts its liver protective effects via mAChR4-induced GAP formation.

### Pharmacological Manipulation of GAPs Alleviates Ethanol-Induced Liver Injury

Based on these findings, a therapeutic approach was developed, administering a mAChR4 PAM, VU0467154, to stimulate intestinal GAP formation. WT mice fed an ethanol containing diet for 10 weeks, were treated with VU0467154 during the last 29 days. VU0467154 has no gastrointestinal motility side effects and excellent oral bioavailability (Bubser et al., 2014; Pancani et al., 2015; Gould et al., 2018). VU0467154 treatment reduced ethanol-induced GAP closure (FIGS. 4A-4B), and protected mice from ethanol-induced liver injury (FIGS. 4C-4D), steatosis (FIGS. 4E-4F) and inflammation (FIG. 4G). mAChR4 PAM treatment did not affect intestinal ethanol absorption, as pair-fed mice had similar blood alcohol levels (FIGS. 9A-B). Frequencies of LP-APCs (CD11c⁺, MHCII⁺) and CD103⁺ CD11b⁺ DC populations in ethanol fed mice were increased by VU0467154 treatment (FIGS. 4H-4K), which also induced intestinal Reg3g, Reg3b and IL-10 expression (FIGS. 4L-4O) and prevented ethanol mediated bacterial translocation to MLN and liver (FIG. 4P-4Q). These results indicate that pharmacological manipulation of mAChR4 reduced ethanol-induced steatohepatitis.

### DISCUSSION

This study reveals a balance between physical barrier function and a protective intestinal immune response. Chronic alcohol increases intestinal mucin production, but this comes at the expense of GAP closing and improper training of intestinal immunity. LP-APCs adjacent to GAPs induce specific protective immune responses that control intestinal homeostasis (McDole et al., 2012; Knoop et al., 2015; Knoop et al., 2017a; Knoop et al., 2016; Kulkarni et al., 2018). Regenerative responses are important after intestinal injury or microbial infection to maintain barrier integrity and prevent translocation of intestinal bacteria (Medzhitov, 2008; Ben-Neriah & Karin, 2011). gp130 signaling stimulates regeneration and regulates differentiation of the gastrointestinal barrier through YAP and Notch activation (Taniguchi et al., 2015; Gregorieff et al., 2015; Yu et al., 2018; Todoric et al., 2020; Romera-Hernandez et al., 2020). Only very recently it was determined that inhibition of a dynamic neuroimmune circuit by activation of VIP receptor type 2 (VIPR2) in the intestine triggers IL-22 secretion by ILC3 and increases Reg3g mRNA in ileum (Talbot et al., 2020). Our findings add to the complex neuronal regulation of antibacterial defenses. Intestinal IL6ST/gp130 controls cholinergic signaling by upregulating mAChR4 on GC to induce GAP formation (FIG. 6). Therefore, IL6ST/gp130 signaling enhances the GC response to acetylcholine released by GC-innervating parasympathetic neurons and possibly other cellular sources.

Ethanol therefore causes liver disease not only via direct toxic effects on liver cells, but also by suppressing the intestinal immune system and allowing enteric bacteria to translocate to the liver, underscoring the importance of the gut-liver axis for ALD pathogenesis. Consistent with this finding, pharmacological manipulation of mAChR4 with a PAM to induce small intestinal GAPs was associated with regulation of APCs, production of IL-22, induction of Reg3 lectins, prevention of bacterial translocation and amelioration of ALD. Interestingly, mAChR4 gene and protein expression are modulated in brain regions of AUD patients. The number of muscarinic receptors increases in several brain regions in the absence of alcohol during withdrawal (Nordberg & Walström, 1992). Systemic administration of VU0467154 reduced ethanol seeking and consumption in a rodent model (Walker et al., 20200. Alcohol consumption was not measured in the mice, as they were pair-fed and received the same amount of ethanol-containing diet. Nevertheless, pharmacological manipulation of mAChR4 could be a potential approach for preventing not only ALD progression, but also for treating AUD. This is an example of the efficacy of intestinal mAChR4 activation for treating diseases outside the gastrointestinal tract. Gp130 agonists and mAChR4 PAM might be therapeutic agents for alcohol-related liver disease.

TABLE 1
Demographic and laboratory results of non-alcoholic controls
and patients with alcohol use disorder (AUD).

| | | | Alcohol use disorder | |
|---|---|---|---|---|
| Variables | Controls (n = 8) | | (n = 15) | |
| Gender (% male), n (%) | 3 | (37.5) | 11 | (73.3) |
| Age (years) | 37.5 | (24.0-57.0) | 44.0 | (27.0-61.0) |
| Weight (kg) | 65 | (51.0-88.0) | 72.0 | (49.0-107.5) |
| BMI (kg/m2) | 22.3 | (19.8-28.1) | 22.4 | (18.0-33.9) |

| | | |
|---|---|---|
| Bilirubin (mg/dL) | 0.4 | (0.2-3.0) |
| AST (IU/L) | 36.0 | (14.0-273.0) |
| ALT (IU/L) | 37.0 | (14.0-133.0) |
| Albumin (g/L) | 48.0 | (32.0-57.5) |
| INR | 0.9 | (0.9-1.2) |
| GGT (IU/L) | 120.0 | (11.0-3460.0) |
| ALP (IU/L) | 83.0 | (41.0-355.0) |
| CAP | 272.0 | (208.0-382.0) |
| FibroScan (Kpa) | 6.1 | (3.1-72.8) |

Median values are represented with range in parentheses for continuous variables or percentage in parentheses for categorical variables. Percentages are calculated based on the number of patients in each group.

ALP, Alkaline phosphatase, ALT, alanine aminotransferase, AST, aspartate aminotransferase; AUD, alcohol use disorder; BMI, body mass index; CAP, Controlled attenuation parameter; GGT, gamma-glutamyl transferase; INR, international normalized ratio.

TABLE 2
Liver disease severity in patients with alcohol use disorder (AUD)

| Liver disease severity | Alcohol use disorder (n = 15) | |
|---|---|---|
| No liver disease, n (%) | 3 | (20) |
| Steatosis, n (%) | 5 | (33.3) |
| Steatohepatitis, n (%) | 2 | (13.3) |
| Steatofibrosis, n (%) | 3 | (20) |
| Unclassified, n (%) | 2 | (13.3) |

Percentages are represented in parentheses.

TABLE 3
Sequences of qPCR primers

| Gene | primer | sequence |
|---|---|---|
| Mouse 18S | F | 5′-AGTCCCTGCCCTTTGTACACA-3′ (SEQ ID |
| | | NO: 1) |
| | R | 5′-CGATCCCAGGGCCTCACTA-3′ (SEQ ID |
| | | NO: 2) |
| Mouse Chrm4 | F | 5′-ATGGCGAACTTCACACCTGTC-3′ (SEQ ID |
| | | NO: 3) |
| | R | 5′-CTGTCGCAATGAACACCATCT-3′ (SEQ ID |
| | | NO: 4) |
| Mouse Cxcl 1 | F | 5′-TGCACCCAAACCGAAGTC-3′ (SEQ ID |
| | | NO: 5) |
| | R | 5′-GTCAGAAGCCAGCGTTCACC-3′ (SEQ ID |
| | | NO: 6) |
| Mouse Ccl2 | F | 5′-ATTGGGATCATCTTGCTGGT-3′ (SEQ ID |
| | | NO: 7) |
| | R | 5′-CCTGCTGTTCACAGTTGCC-3′ (SEQ ID |
| | | NO: 8) |
| Mouse Cxcl5 | F | 5′-TGATCCCTGCAGGTCCACA-3′ (SEQ ID |
| | | NO: 9) |
| | R | 5′-CTGCGAGTGCATTCCGCTTA-3′ (SEQ ID |
| | | NO: 10) |
| Mouse Tbp | F | 5′-GTGAAGGGTACAAGGGGGTG-3′ (SEQ ID |
| | | NO: 11) |
| | R | 5′-ACATCTCAGCAACCCACACA-3′ (SEQ ID |
| | | NO: 12) |
| Bacteria 16S | F | 5′-GTG STG CAY GGY TGT CGT CA-3′ (SEQ |
| | | ID NO: 14 |
| | R | 5′-ACG TCR TCC MCA CCT TCC TC-3′ (SEQ |
| | | ID NO: 15) |

### REFERENCES

Aymanns et al., PLoS One, 6:e19822 (2011).
Ben-Neriah & Karin, Nat. Immunol., 12:715 (2011).
Bruellman & Llorente, Int. J. Biol. Sci., 17:307 (2021).
Bubser et al., ACS Chem. Neurosci., 5:920 (2014).
Caporaso et al., Proc. Nat. Acad. Sci. USA 108:4516 (2011).
Chen et al., Gastroenterology 148:203 (2015).
Collaborators, Lancet., 392:1015 (2018).
Denning et al., J. Immunol. 187:733 (2011).
Duan et al., Nature 575:505 (2019).
Fouts et al., J. Hepatol., 56:1283 (2012).
Gould et al., Neuropharmacology 128:492 (2018).
Gregorieff et al., Nature, 526:715 (2015).
Haas et al., Genome Res., 21:494 (2011).
Hartmann et al., Hepatology, 58:108 (2013).
Hong et al., Oncogene, 21:32 (2002).
Knoop et al., Gut. Microbes. 8:400 (2017).
Knoop et al., Gut., 65:1100 (2016).
Knoop et al., Mucosal. Immunol. 8:198 (2015).
Knoop et al., Sci. Immunol. 2:_(2017b).
Kuhn et al., Mucosal. Immunol., 11:357 (2018).
Kulkarni et al., Mucosal. Immunol., 11:1103 (2018).
Kulkarni et al., Mucosal. Immunol., 13:271 (2020).
Lang & Schnabl, Cell Host Microbe., 28:233 (2020).
Lee et al., JAMA Intern. Med. 179:340 (2019).
Llorente et al., Nat. Commun., 8:837 (2017).
Maeda et al., FEMS Immunol. Med. Microbiol. 39:81 (2003).
McDole et al., Nature, 483:345 (2012).
Medzhitov, Nature, 454:428 (2008).
Nordberg & Wahlström, J. Neural. Transmission/General Section JNT, 88:199 (1992).
Pancani et al., Proc. Natl. Acad. Sci. USA 112:14078 (2015).
Romera-Hernandez et al., Cell. Rep., 30:37 (2020).
Sato et al., Nature 459:262 (2009).
Sheron et al., Clin. Exp. Immunol., 84:449 (1991).
Talbot et al., Nature, 579:575 (2020).
Taniguchi et al., Nature, 519:57 (2015).
Todoric et al., Nat. Metab., 2:1034 (2020).
Velcich et al., Science, 295:1726 (2002).
Walker et al., Biol. Psychiatry, 88:898 (2020).
Wang et al., Cell Host Microbe., 19:227 (2016).
Yan et al., Hepatology, 53:96 (2011).
Yu et al., Cell Stem Cell, 23:46 (2018).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to prevent, inhibit or treat liver disease in a mammal, comprising administering to the mammal an effective amount of a composition comprising one or more mAChR4 positive allosteric modulators.

2. A method to prevent, inhibit or treat microbial translocation in a mammal, comprising administering to the mammal an effective amount of a composition comprising one or more mAChR4 positive allosteric modulators.

3. A method to stimulate intestinal GAP formation or increase tolerogenic LP-APCs and Reg3 expression in a mammal, comprising administering to the mammal an effective amount of a composition comprising one or more mAChR4 positive allosteric modulators.

4. A method to enhance intestinal immune tolerance in a mammal, comprising administering to the mammal an effective amount of a composition comprising one or more mAChR4 positive allosteric modulators.

5. The method of any one of claims 1 to 4, wherein the mammal is a human.

6. The method of any one of claims 1 to 5, wherein the composition is systemically administered.

7. The method of any one of claims 1 to 5, wherein the composition is orally administered.

8. The method of any one of claims 1 to 5, wherein the composition is parenterally administered.

9. The method of any one of claims 1 to 8, wherein the composition further comprises a pharmaceutically acceptable carrier.

10. The method of any one of claims 1 to 9, wherein the composition is a sustained realse composition.

11. The method of any one of claims 1 to 10, wherein the mammal has alcoholic liver disease.

12. The method of any one of claims 1 to 11, wherein multiple doses of the composition are administered.

13. The method of any one of claims 1 to 12 further comprising administering a Gp130 agonist.

14. The method of claim 13 wherein the Gp130 agonist comprises an antibody.

15. The method of claim 13 wherein the Gp130 agonist comprises CAS 339303-87-6 (UCLA GP130 2).

16. The method of any one of claims 1 or 4 to 15, wherein the amount reduces ethanol-induced steatohepatitis.

17. The method of any one of claims 1 or 4 to 15, wherein the amount reduces ethanol-induced liver injury.

18. The method of any one of claims 1 or 4 to 15, wherein the amount reduces steatosis.

19. The method of any one of claims 1 to 18, wherein the modulator contains a 3-aminothiophene-2-carboxamide moiety.

20. The method of any one of claims 1 to 19, wherein the modulator has a structure according to Formula I, or a pharmaceutically acceptable salt thereof: 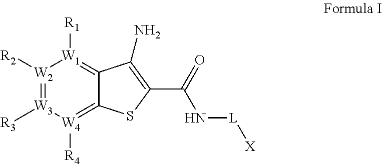
wherein
each of w₁, w₂, w₃, and w₄ is independently C or N;
each of R₁, R₂, R₃, and R₄ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH₂-heterocycloalkyl, —CH₂-aryl, or —CH₂-heteroaryl, CHF₂, CH₂F, or CF₃;
L is absent or CH₂;
X is a cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

21. The method of any one of claims 1 to 19, wherein the modulator has a structure according to Formula I, or a pharmaceutically acceptable salt thereof: 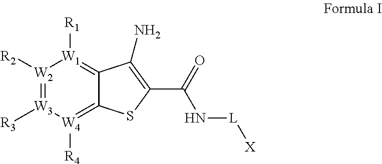
wherein
each of w₁, w₂, w₃, and w₄ is independently C or N;
each of R₁, R₂, R₃, and R₄ is independently H, F, Cl, Br, alkyl, ORₐ, or a lone pair if the group to which it is attached is N;
Rₐ is H, alkyl, or —CH₂-pyridine;
L is absent or CH₂;
X is a phenyl, pyridine or cyclopropyl, each of which may be optionally substituted with one or more group selected from Y, or two Y are linked so as to form a fused 1,3-dioxolane together with the atoms to which they are attached; and
Y is independently F, Cl, Br, CHF₂, CH₂F, CF₃, SO2R_{b}, SOR_{b}, SO, SR_{b}, SF₅, phenyl optionally substituted by Z, pyridine optionally substituted by Z, pyrimidine optionally substituted by Z, pyridazine optionally substituted by Z, pyrazine optionally substituted by Z,
Z is methyl, F, or OMe; and
R_{b} is H, alkyl, cycloalkyl, CHF₂, CH₂F, or CF₃.

22. The method of any one of claims 1 to 19, wherein w₁ and w₂ are each C and w₃ and w₄ are each N.

23. The method of claim 22, wherein w₁ and w₂ are each C and w3 and w4 are each N.

24. The method of any one of claims 1 to 23, wherein w₁, w₂, and w₃ are each C, and w₄ is N.

25. The method of claim 20, wherein w₁, w₂, and w₃ are each C, and w₄ is N.

26. The method of any one of claims 1 to 19, wherein the modulator has a structure according to Formula II, or a pharmaceutically acceptable salt thereof: 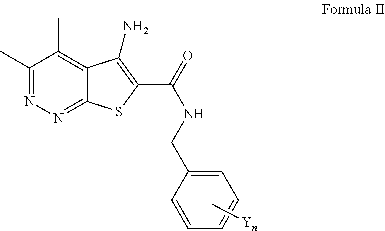
wherein
Y is independently F, Cl, Br, CHF₂, CH₂F, CF₃, SO2R_{b}, SOR_{b}, SO, SR_{b}, SF₅, phenyl optionally substituted by Z, pyridine optionally substituted by Z, pyrimidine optionally substituted by Z, pyridazine optionally substituted by Z, pyrazine optionally substituted by Z,
Z is methyl, F, or OMe;
R_{b} is H, alkyl, cycloalkyl, CHF₂, CH₂F, or CF₃; and
wherein n is 0-3.

27. The method of any one of claims 1 to 19, wherein the modulator has a structure according to Formula III, or a pharmaceutically acceptable salt thereof: 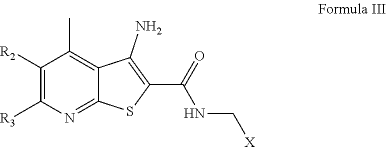
wherein
each of R₂, R₃, and R₄ is independently H, F, Cl, alkyl, ORₐ;
Rₐ is alkyl, or —CH₂-pyridine;
X is a phenyl or pyridine, each of which may be optionally substituted with one or more group selected from Y, or two Y are linked so as to form a fused 1,3-dioxolane together with the atoms to which they are attached; and
Y is independently F, Cl, Br, CHF₂, CH₂F, CF₃, OR_{b}, SO2R_{b}, SOR_{b}, SO, SR_{b}, SF₅, phenyl optionally substituted by Z, pyridine optionally substituted by Z, pyrimidine optionally substituted by Z, pyridazine optionally substituted by Z, pyrazine optionally substituted by Z,
Z is methyl, F, or OMe; and
R_{b} is H, alkyl, cycloalkyl, CHF₂, CH₂F, or CF₃.

28. The method of any one of claims 1 to 19, wherein the modulator has a structure according to Formula IV, or a pharmaceutically acceptable salt thereof: 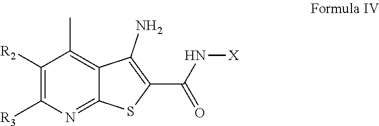
wherein each of R₂, R₃, and R₄ is independently H, F, Cl, or alkyl; and X is alkyl, cycloalkyl, aryl, heterocycloalkyl, or heteroaryl.

29. The method of any one of claims 1 to 19, wherein the modulator has a structure according to Formula V, or a pharmaceutically acceptable salt thereof: 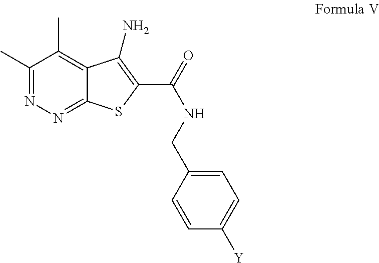
wherein Y is independently F, Cl, Br, CHF₂, CH₂F, CF₃, SO2R_{b}, SOR_{b}, SO, SR_{b}, SF₅; and R_{b} is H, alkyl, cycloalkyl, CHF₂, CH₂F, or CF₃.

30. The method of any one of claims 1 to 19, wherein the modulator has a structure according to Formula VI, or a pharmaceutically acceptable salt thereof: 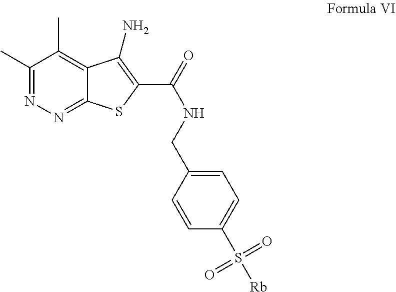
wherein R_{b} is H, alkyl, cycloalkyl, CHF₂, CH₂F, or CF₃.

31. The method of any one of claims 1 to 19, wherein the modulator has the structure: 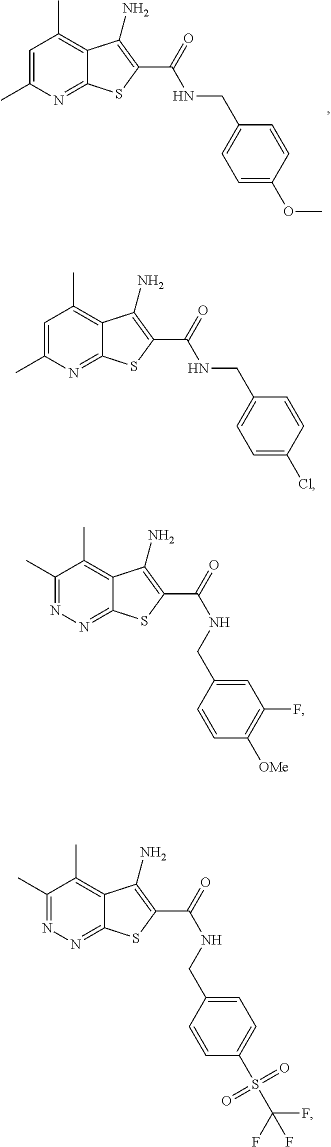 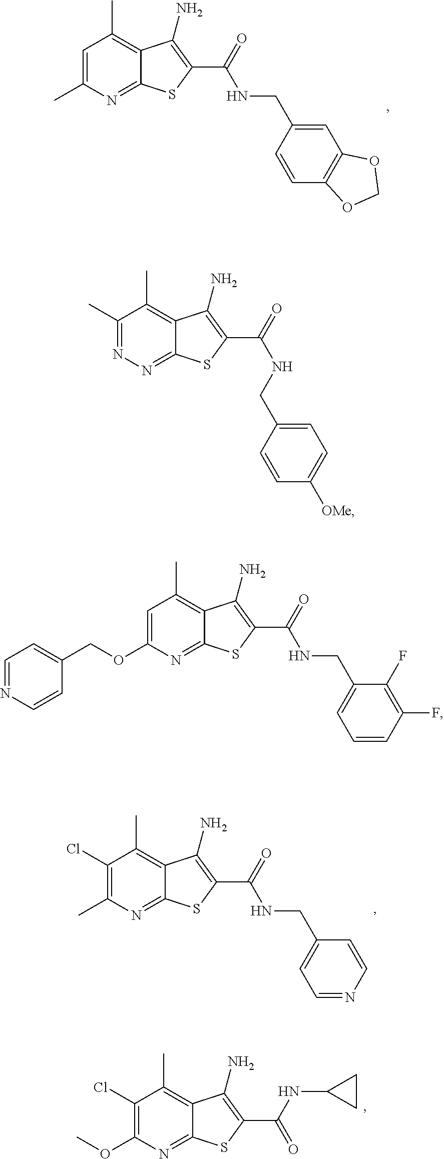 [SMILES: Cc1cc(C)c2c(N)c(C(=O)NCc3ccc4c(c3)OCO4)sc2n1 ; COc1ccc(CNC(=O)c2sc3nnc(C)c(C)c3c2N)cc1 ; Cc1cc(OCc2ccncc2)nc2sc(C(=O)NCc3cccc(F)c3F)c(N)c12 ; Cc1nc2sc(C(=O)NCc3ccncc3)c(N)c2c(C)c1Cl ; COc1nc2sc(C(=O)NC3CC3)c(N)c2c(C)c1Cl] 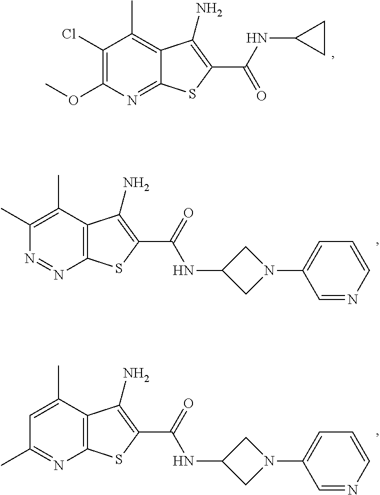 [SMILES: COc1nc2sc(C(=O)NC3CC3)c(N)c2c(C)c1Cl ; Cc1nnc2sc(C(=O)NC3CN(c4cccnc4)C3)c(N)c2c1C ; Cc1cc(C)c2c(N)c(C(=O)NC3CN(c4cccnc4)C3)sc2n1]
or a pharmaceutically acceptable salt thereof.

32. The method of any one of claims 1 to 4, wherein the modulator has a structure according to Formula VII, or a pharmaceutically acceptable salt thereof: 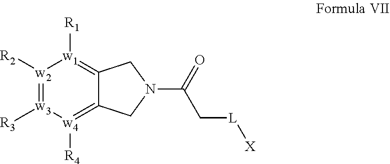
wherein
each of w₁, w₂, w₃, and w₄ is independently C or N;
each of R₁, R₂, R₃, and R₄ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
L is absent or CH₂;
X is a cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

33. The method of any one of claims 1 to 4, wherein the modulator has a structure according to Formula VIII, or a pharmaceutically acceptable salt thereof: 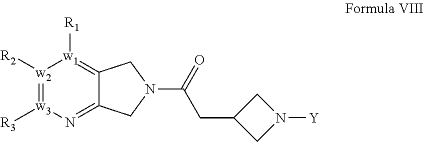
each of w₁, w₂, and w₃, is independently C or N;
each of R₁, R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

34. The method of any one of claims 1 to 4, wherein the modulator has a structure according to Formula IX, or a pharmaceutically acceptable salt thereof: 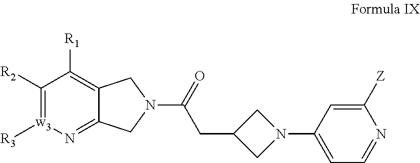
each of w₁, w₂, and w₃, is independently C or N;
each of R₁, R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

35. The method of any one of claims 1 to 4, wherein the modulator is a tricyclic antidepressant (TCA) or has a structure according to Formula Xa, or a pharmaceutically acceptable salt thereof: 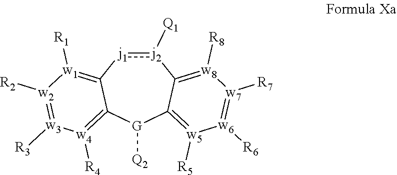
wherein
each of w₁, w₂, w₃, w₄, w₅, w₆, w₇, and w₈, is independently C or N;
each of R₁, R₂, R₃, R₄, R₅, R₆, R₇, and R₈ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
each of j₁, j₂, and G is independently CH, CH₂, N, O, S, SO2, SO, or NRₐ;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
one of Q₁ and Q₂ is X and the other is H, or a lone pair if the group to which it is attached is N, O, or S;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached, and when X is attached to G the bond between X and G is a single bond or a double bond;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

36. The method of any one of claims 1 to 4, wherein the modulator has a structure according to Formula Xb, or a pharmaceutically acceptable salt thereof: 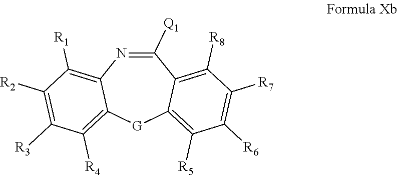
wherein
each of R₁, R₂, R₃, R₄, R₅, R₆, R₇, and R₈ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro;
G is independently CH₂, O, S, SO2, SO, CRₐ, or NRₐ;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q₁ is X;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached, and when X is attached to G the bond between X and G is a single bond or a double bond;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

37. The method of any one of claims 1 to 4, wherein the modulator has a structure according to Formula XI, or a pharmaceutically acceptable salt thereof: 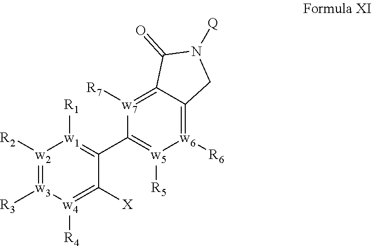
wherein
each of w₁, w₂, w₃, w₄, w₅, w₆, and w₇, is independently C or N;
each of R₁, R₂, R₃, R₄, R₅, R₆, and R₇, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q is H, alkyl, cycloalkyl, heterocycloalkyl, aryl, or heteroaryl, each of which may be optionally radiolabeled;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

38. The method of any one of claims 1 to 4, wherein the modulator has a structure according to Formula XII, or a pharmaceutically acceptable salt thereof: 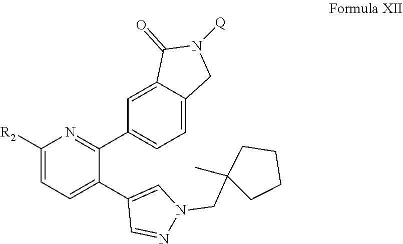
wherein
each R₂ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q is H, alkyl, cycloalkyl, heterocycloalkyl, aryl, or heteroaryl, each of which may be optionally radiolabeled.

39. The method of any one of claims 1-4, wherein the modulator has a structure according to Formula XIII, or a pharmaceutically acceptable salt thereof: 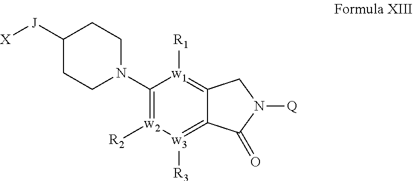
wherein
each of w₁, w₂, and w₃, is independently C or N;
each of R₁, R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Q is H, alkyl, cycloalkyl, heterocycloalkyl, aryl, or heteroaryl, each of which may be optionally radiolabeled;
J is CHRₐ, O, S, NRₐ, or absent;
X is alkyl, alkenyl, cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

40. The method of any one of claims 1 to 4, wherein the modulator has a structure according to Formula XIV, or a pharmaceutically acceptable salt thereof: 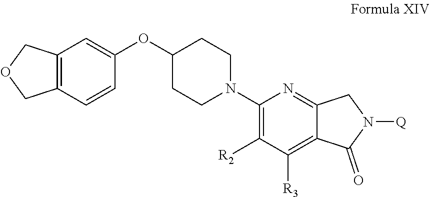
wherein
each of R₂, and R₃, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃; and
Q is H, alkyl, cycloalkyl, heterocycloalkyl, aryl, or heteroaryl, each of which may be optionally radiolabeled.

41. The method of any one of claims 1 to 4, wherein the modulator has a structure according to Formula XV, or a pharmaceutically acceptable salt thereof: 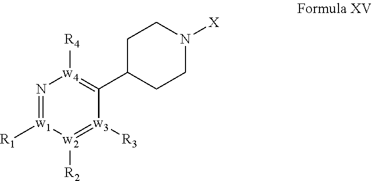
wherein
each of w₁, w₂, w₃, and w₄, is independently C or N;
each of R₁, R₂, R₃, and R₄, is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
X is cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

42. The method of any one of claims 1 to 4, wherein the modulator has a structure according to Formula XVI, or a pharmaceutically acceptable salt thereof: 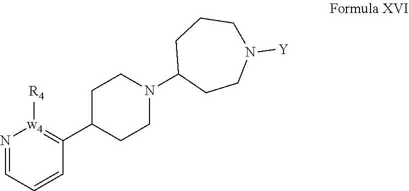
wherein
w₄ is independently C or N;
R₄ is independently H, F, Cl, Br, I, alkyl, cycloalkyl, heterocycloalkyl, aryl, or heteroaryl, CHF₂, CH₂F, CF₃, CORₐ, CONHRₐ, COORₐ, ORₐ, OCORₐ, OCONHRₐ, NHRₐ NHCORₐ, NHCONHRₐ, NHCOORₐ, NHRₐ, N(Rₐ)₂, NHSO₂Rₐ, SO2Rₐ, SORₐ, SRₐ, SF₅, cyano, or nitro, or a lone pair if the group to which it is attached is N;
Rₐ is H, alkyl, cycloalkyl, heterocycloalkyl, aryl, or heteroaryl, —CH₂-cycloalkyl, —CH2-heterocycloalkyl, —CH2-CO-heterocycloalkyl, —CH₂-aryl, or —CH₂-heteroaryl, CHF₂, CH₂F, or CF₃;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z; and
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

43. The method of any one of claims 1 to 4, wherein the modulator has a structure according to Formula XVII, or a pharmaceutically acceptable salt thereof: 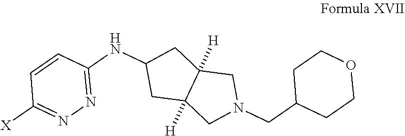
wherein
X is cycloalkyl, heterocycloalkyl, aryl, or heteroaryl, each of which may be optionally substituted with one or more Y, or two Y are linked so as to form a fused cycloalkyl, heterocycloalkyl, aryl, or heteroaryl together with the atoms to which they are attached;
Y is independently F, Cl, Br, I, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, nitro, alkyl optionally substituted by Z, cycloalkyl optionally substituted by Z, heterocycloalkyl optionally substituted by Z, aryl optionally substituted by Z, or heteroaryl optionally substituted by Z;
Z is independently H, F, Cl, Br, I, alkyl, cycloalkyl, heterocycloalkyl, CHF₂, CH₂F, CF₃, COR_{b}, CONHR_{b}, COOR_{b}, OR_{b}, OCOR_{b}, OCONHR_{b}, NHR_{b}, NHCOR_{b}, NHCONHR_{b}, NHCOOR_{b}, NHR_{b}, N(R_{b})₂, NHSO₂R_{b}, SO2R_{b}, SOR_{b}, SO, SR_{b}, SF₅, cyano, or nitro; and
R_{b} is H, alkyl, cycloalkyl, CHF₂, CH₂F, or CF₃.

44. The method of any one of claims 1 to 4, wherein the modulator has a structure according to Formula XVIII, or a pharmaceutically acceptable salt thereof: 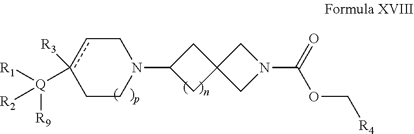
wherein
n is 1 or 2
p is 0, 1 or 2
Q is a five, six or seven membered monocyclic heterocyclic ring containing 1, 2, 3 or 4 heteroatom ring members selected from N, O and S;
R1 is selected from hydrogen; fluorine; chlorine; bromine; cyano; oxo; hydroxy; OR5; NR5R6; COR5; COOR5; OCOR5; NR7COR5; CONR5R6; NR7CONR5R6; NR7COOR5; OCONR5R6; SR5; SOR5 and S02R5; a Ci.e non-aromatic hydrocarbon group which is optionally substituted with one to six fluorine atoms and wherein one or two, but not all, carbon atoms of the hydrocarbon group may optionally be replaced by a heteroatom selected from O, N and S and oxidized forms thereof; and an optionally substituted 5- or 6-membered ring containing 0, 1, 2 or 3 heteroatoms selected from O, N and S and oxidized forms thereof;
R2 is selected from hydrogen; fluorine; chlorine; bromine; cyano; hydroxy; methoxy; OR5; NR5R6; COR5; COOR5; OCOR5; NR7COR5; CONR5R6;
NR7CONR5R6; NR7COOR5; OCONR5R6; SR5; SOR5 and S02R5; and a d.6 non-aromatic hydrocarbon group; or R1 and R2 can be joined together to form a 6 membered fused aromatic ring;
R9 is selected from hydrogen, CH3, CH2OH, CH(CH3)OH, C(CH3)2OH and COOCH3;
R3 is selected from hydrogen; fluorine; cyano; hydroxy; amino; and a Ci_9 non-aromatic hydrocarbon group which is optionally substituted with one to six fluorine atoms and wherein one, two or three, but not all, carbon atoms of the hydrocarbon group may optionally be replaced by a heteroatom selected from O, N and S and oxidized forms thereof; R4 is a hydrogen or a Ci_6 non-aromatic hydrocarbon group which is optionally substituted with one to six fluorine atoms and wherein one or two, but not all, carbon atoms of the hydrocarbon group may optionally be replaced by a heteroatom selected from O, N and S and oxidised forms thereof;
R5, R6 and R7 are the same or different and each is independently selected from hydrogen, a non-aromatic Ci_4 hydrocarbon group optionally substituted with one or more fluorine atoms, or a group of formula CH2N(Ra)COORb;
Ra is selected from hydrogen and a non-aromatic Ci_4 hydrocarbon group; Rb is a non-aromatic Ci_4 hydrocarbon group which is optionally substituted with one or more groups selected from fluorine; chlorine; bromine; cyano; hydroxy; methoxy; amino; or a cycloalkyl, heterocycloalkyl, aryl or heteroaryl group; and the dotted line indicates an optional second carbon-carbon bond, provided that when a second carbon-carbon bond is present, then R3 is absent.

45. The method of any one of claims 1 to 4, wherein the modulator has the structure: VU0467485, ML173, VU0448088, VU0464090, or a pharmaceutically acceptable salt thereof.

46. The method of any one of claims 1 to 4, wherein the modulator has the structure: VU0467154, VU0152100, VU0152099, LY2033298, or VU010010, or a pharmaceutically acceptable salt thereof.

47. The method of any one of claims 1 to 4, wherein the modulator has the structure: PT-1148, ([11C]MK-6884, MK-4710, PT-6950, LY2033298, PT-3763, VU0448088 [ML253], VU0467485 (AZ13713945), or CVL-231, or a pharmaceutically acceptable salt thereof.

48. The method of any one of claims 1 to 4, wherein the modulator has the structure: VU0152100, LY2033298, VU6013720, VU6021302, VU6021625, LY 2119620, VU 0467485, VU 10010, WO2017021728 A1, McN-A-343 (C7041), Xanomeline (X2754), Thiochrome, Vanderbilt's VU0010010, LY2119620, VU0152099, ML173, VU0448088 [ML253], VU0467154, VU0467485/AZ13713945, VU0409524, VU6002703, VU6003130, VU6005877, ([11C]MK-6884], MK-4710, CVL-231 (NCT04136873), VU0238441, HTL-9936, a dihydroquinazolinone, a n-substituted 7-azaindoline, a N-substituted oxindole, HTL0016878, a 1,2,3-thiadiazole, or pyrazine.

49. The method of any one of claims 1 to 4, wherein the modulator has the structure: 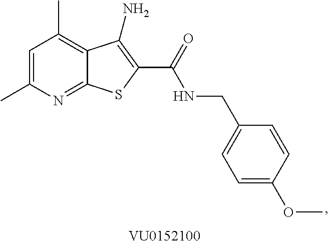
or a prodrug or pharmaceutically acceptable salt thereof.

50. The method of any one of claims 1 to 4, wherein the modulator has the structure: 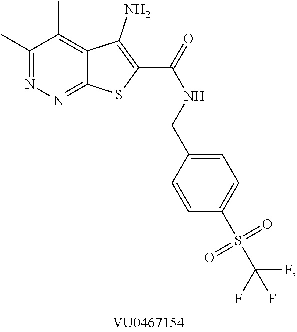 [SMILES: Cc1nnc2sc(C(=O)NCc3ccc(S(=O)(=O)C(F)(F)F)cc3)c(N)c2c1C]
or a prodrug or pharmaceutically acceptable salt thereof.

51. The method of any one of claims 1 to 4, wherein the modulator has the structure: 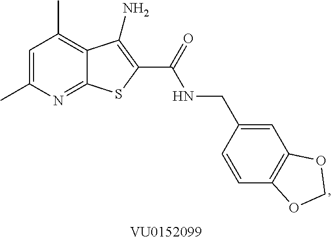 [SMILES: Cc1cc(C)c2c(N)c(C(=O)NCc3ccc4c(c3)OCO4)sc2n1]
or a prodrug or pharmaceutically acceptable salt thereof.

52. The method of any one of claims 1 to 4, wherein the modulator has the structure: 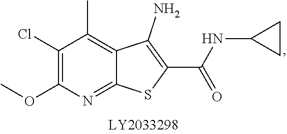 [SMILES: COc1nc2sc(C(=O)NC3CC3)c(N)c2c(C)c1Cl]
or a prodrug or pharmaceutically acceptable salt thereof.

53. The method of any one of claims 1 to 4, wherein the modulator has the structure: 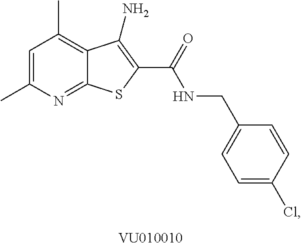 [SMILES: Cc1cc(C)c2c(N)c(C(=O)NCc3ccc(Cl)cc3)sc2n1]
or a prodrug or pharmaceutically acceptable salt thereof.

54. The method of any one of claims 1 to 4, wherein the modulator has the structure: 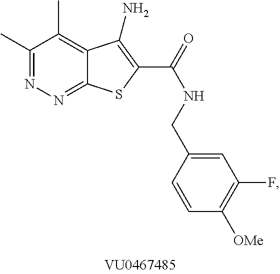 [SMILES: COc1ccc(CNC(=O)c2sc3nnc(C)c(C)c3c2N)cc1F]
or a prodrug or pharmaceutically acceptable salt thereof.

55. The method of any one of claims 1 to 4, wherein the modulator has the structure: 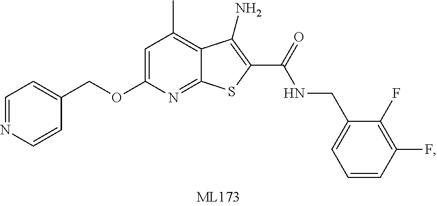 [SMILES: Cc1cc(OCc2ccncc2)nc2sc(C(=O)NCc3cccc(F)c3F)c(N)c12]
or a prodrug or pharmaceutically acceptable salt thereof.

56. The method of any one of claims 1 to 4, wherein the modulator has the structure: 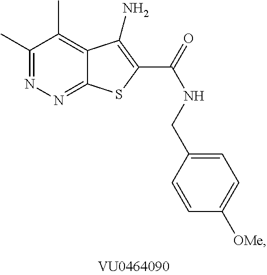 [SMILES: COc1ccc(CNC(=O)c2sc3nnc(C)c(C)c3c2N)cc1]
or a prodrug or pharmaceutically acceptable salt thereof.

57. The method of any one of claims 1 to 4, wherein the modulator has the structure: 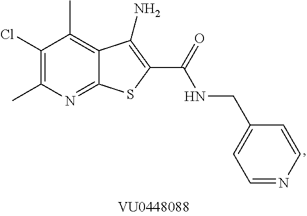 [SMILES: Cc1nc2sc(C(=O)NCc3ccncc3)c(N)c2c(C)c1Cl]
or a prodrug or pharmaceutically acceptable salt thereof.

58. The method of any one of claims 1 to 4, wherein the modulator has the structure: 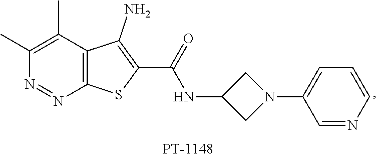 [SMILES: Cc1nnc2sc(C(=O)NC3CN(c4cccnc4)C3)c(N)c2c1C]
or a prodrug or pharmaceutically acceptable salt thereof.

59. The method of any one of claims 1 to 4, wherein the modulator has the structure: 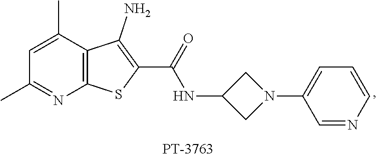 [SMILES: Cc1cc(C)c2c(N)c(C(=O)NC3CN(c4cccnc4)C3)sc2n1]
or a prodrug or pharmaceutically acceptable salt thereof.

60. The method of any one of claims 1 to 4, wherein the modulator has the structure: 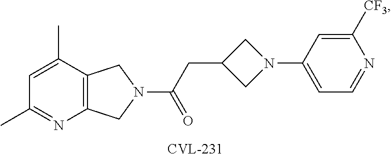 [SMILES: Cc1cc(C)c2c(n1)CN(C(=O)CC1CN(c3ccnc(C(F)(F)F)c3)C1)C2]
or a prodrug or pharmaceutically acceptable salt thereof.

61. The method of any one of claims 1 to 4, wherein the modulator has the structure: 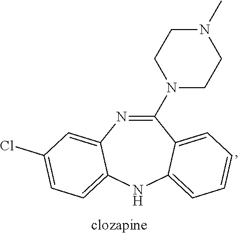 [SMILES: CN1CCN(C2=Nc3cc(Cl)ccc3Nc3ccccc32)CC1]
or a derivative, a prodrug, or pharmaceutically acceptable salt thereof.

62. The method of any one of claims 1 to 4, wherein the modulator has the structure: 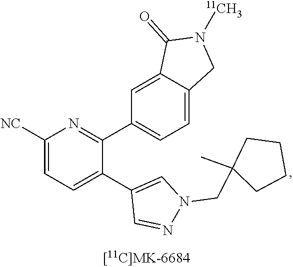
or a prodrug or pharmaceutically acceptable salt thereof.

63. The method of any one of claims 1 to 4, wherein the modulator has the structure: 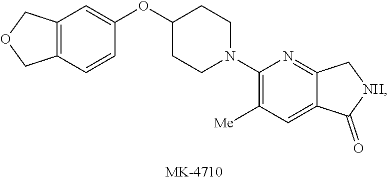 [SMILES: Cc1cc2c(nc1N1CCC(Oc3ccc4c(c3)COC4)CC1)CNC2=O]
or a prodrug or pharmaceutically acceptable salt thereof.

64. The method of any one of claims 1 to 4, wherein the modulator has the structure: 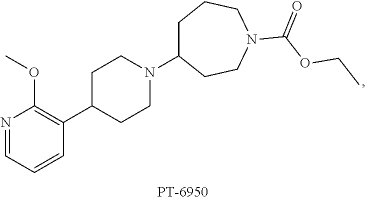 [SMILES: CCOC(=O)N1CCCC(N2CCC(c3cccnc3OC)CC2)CC1]
or a prodrug or pharmaceutically acceptable salt thereof.

65. The method of any one of claims 1 to 4, wherein the modulator has the structure: 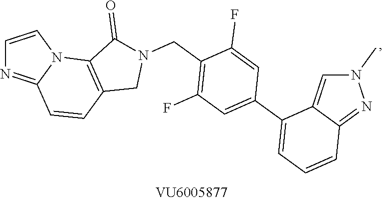 [SMILES: Cn1cc2c(-c3cc(F)c(CN4Cc5ccc6nccn6c5C4=O)c(F)c3)cccc2n1]
or a prodrug or pharmaceutically acceptable salt thereof.

66. The method of any one of claims 1 to 4, wherein the modulator has the structure: 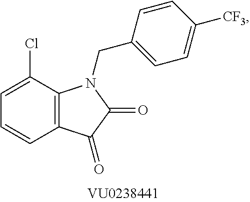 [SMILES: O=C1C(=O)N(Cc2ccc(C(F)(F)F)cc2)c2c(Cl)cccc21]
or a prodrug or pharmaceutically acceptable salt thereof.

67. The method of any one of claims 1 to 4, wherein the modulator has the structure: 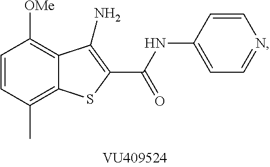 [SMILES: COc1ccc(C)c2sc(C(=O)Nc3ccncc3)c(N)c12]
or a prodrug or pharmaceutically acceptable salt thereof.

68. The method of any one of claims 1 to 4, wherein the modulator has the structure: 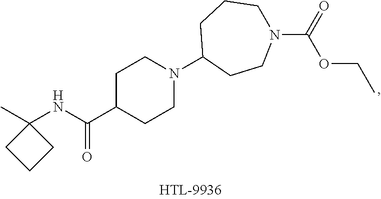 [SMILES: CCOC(=O)N1CCCC(N2CCC(C(=O)NC3(C)CCC3)CC2)CC1]
or a prodrug or pharmaceutically acceptable salt thereof.

69. The method of any one of claims 1 to 4, wherein the modulator has the structure: 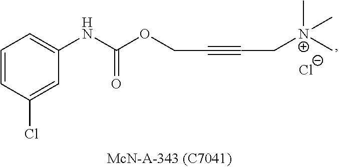 [SMILES: C[N+](C)(C)CC#CCOC(=O)Nc1cccc(Cl)c1 ; [Cl-]]
or a prodrug or pharmaceutically acceptable salt thereof.

70. The method of any one of claims 1 to 4, wherein the modulator has the structure: 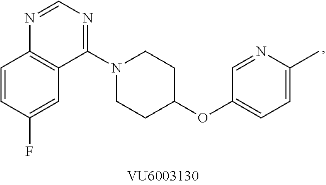 [SMILES: Cc1ccc(OC2CCN(c3ncnc4ccc(F)cc34)CC2)cn1]
or a prodrug or pharmaceutically acceptable salt thereof.

71. The method of any one of claims 1 to 4, wherein the modulator has the structure: 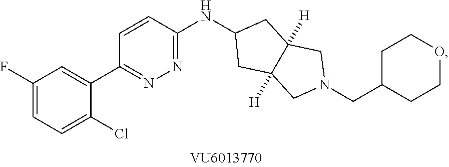 [SMILES: Fc1ccc(Cl)c(-c2ccc(NC3C[C@@H]4CN(CC5CCOCC5)C[C@@H]4C3)nn2)c1]
or a prodrug or pharmaceutically acceptable salt thereof.

72. The method of any one of claims 1 to 4, wherein the modulator has the structure: 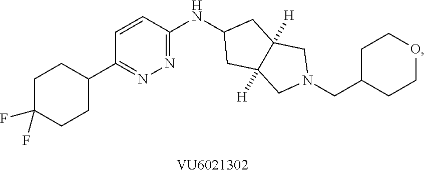 [SMILES: FC1(F)CCC(c2ccc(NC3C[C@@H]4CN(CC5CCOCC5)C[C@@H]4C3)nn2)CC1]
or a prodrug or pharmaceutically acceptable salt thereof.

73. The method of any one of claims 1 to 4, wherein the modulator has the structure: 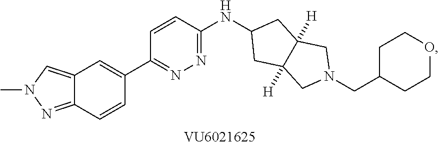 [SMILES: Cn1cc2cc(-c3ccc(NC4C[C@@H]5CN(CC6CCOCC6)C[C@@H]5C4)nn3)ccc2n1]
or a prodrug or pharmaceutically acceptable salt thereof.

74. The method of any one of claims 1 to 4, wherein the modulator has the structure: 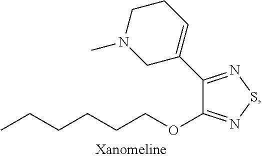 [SMILES: CCCCCCOc1nsnc1C1=CCCN(C)C1]
or a prodrug or pharmaceutically acceptable salt thereof.

75. The method of any one of claims 1 to 4, wherein the modulator has the structure: 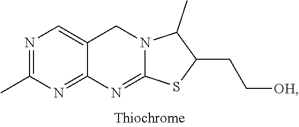 [SMILES: Cc1ncc2c(n1)N=C1SC(CCO)C(C)N1C2]
or a prodrug or pharmaceutically acceptable salt thereof.

76. The method of any one of claims 1 to 4, wherein the modulator has the structure: 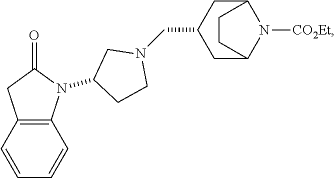
or a prodrug or pharmaceutically acceptable salt thereof.

77. The method of any one of claims 1 to 4, wherein the modulator has the structure: 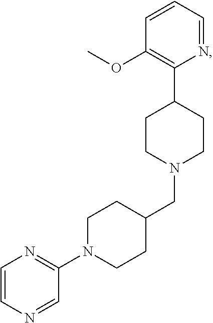 [SMILES: COc1cccnc1C1CCN(CC2CCN(c3cnccn3)CC2)CC1]
or a prodrug or pharmaceutically acceptable salt thereof.

78. The method of any one of claims 1 to 4, wherein the modulator has the structure: 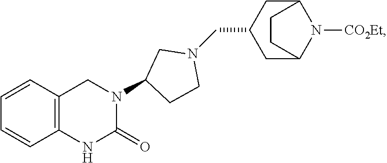
or a prodrug or pharmaceutically acceptable salt thereof.

79. The method of any one of claims 1 to 4, wherein the modulator has the structure: 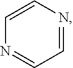
or a prodrug or pharmaceutically acceptable salt thereof.

80. The method of any one of claims 1 to 4, wherein the modulator has the structure: 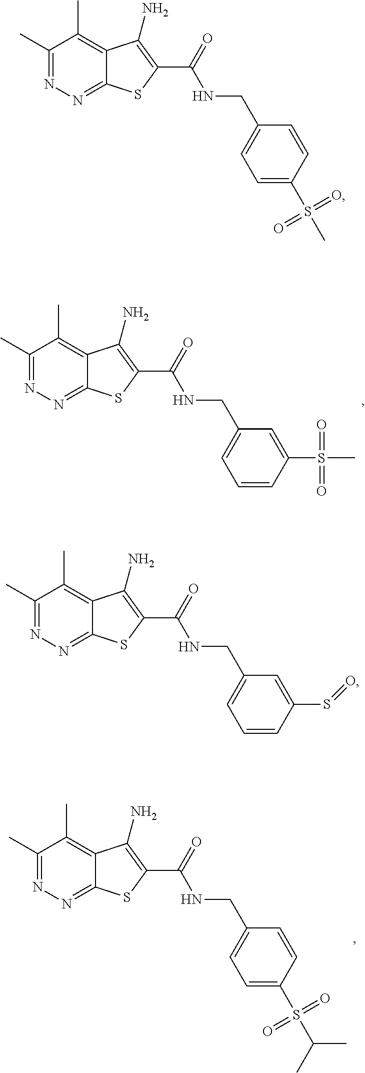 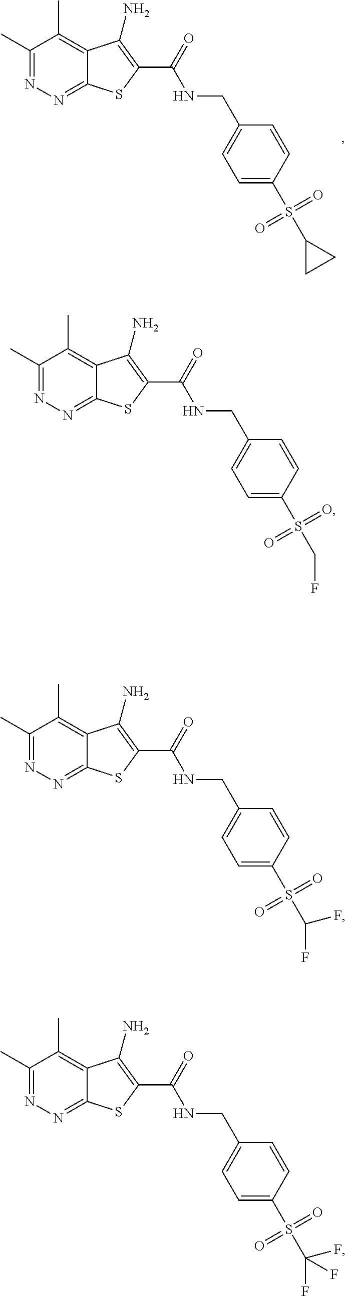 [SMILES: Cc1nnc2sc(C(=O)NCc3ccc(S(=O)(=O)C4CC4)cc3)c(N)c2c1C ; Cc1nnc2sc(C(=O)NCc3ccc(S(=O)(=O)CF)cc3)c(N)c2c1C ; Cc1nnc2sc(C(=O)NCc3ccc(S(=O)(=O)C(F)F)cc3)c(N)c2c1C ; Cc1nnc2sc(C(=O)NCc3ccc(S(=O)(=O)C(F)(F)F)cc3)c(N)c2c1C] 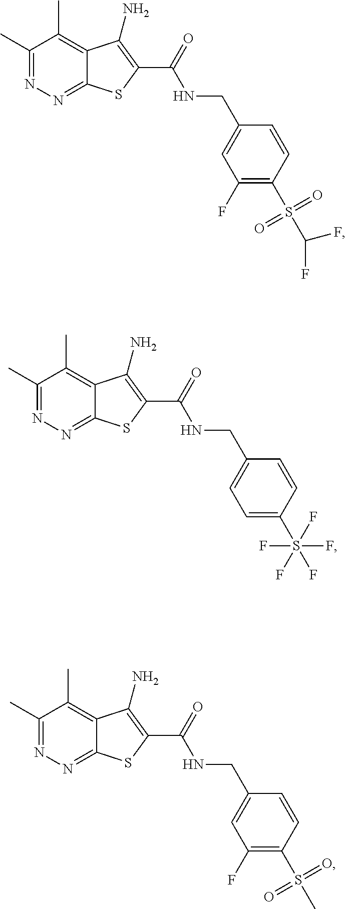 [SMILES: Cc1nnc2sc(C(=O)NCc3ccc(S(=O)(=O)C(F)F)c(F)c3)c(N)c2c1C ; Cc1nnc2sc(C(=O)NCc3ccc(S(F)(F)(F)(F)F)cc3)c(N)c2c1C ; Cc1nnc2sc(C(=O)NCc3ccc(S(C)(=O)=O)c(F)c3)c(N)c2c1C]
or a pharmaceutically acceptable salt thereof.
